(19) United States
(12) Patent Application Publication
(10) Pub. No.: US 2026/0014260 A1
(43) Pub. Date: Jan. 15, 2026

(54) MDM2 DEGRADER

(71) Applicant: Newave Pharmaceutical Inc., Pleasanton, CA (US)

(72) Inventor: Yi Chen, Pleasanton, CA (US)

(21) Appl. No.: 18/865,919

(22) PCT Filed: May 23, 2023
(86) PCT No.: PCT/US2023/023234

§ 371 Date: Nov. 14, 2024

Related U.S. Application Data
(60) Provisional application No. 63/345,172, filed on May 24, 2022.

(51) Int. Cl. A61K 47/55

(52) U.S. Cl. CPC A61K 47/55

(57) ABSTRACT

The disclosure includes compounds of Formula (1) wherein each of R, R₁, R₂, R_{2A}, R₃, R₄, R₅, R₆, R, L₁, L₂, L₃, L₄, L₅, L₆, Q₁, Q₂, Q₃, m, n, r, s, and t, are defined herein. Also disclosed is a method for treating a neoplastic disease, autoimmune disease, and inflammatory disorder with these compounds.

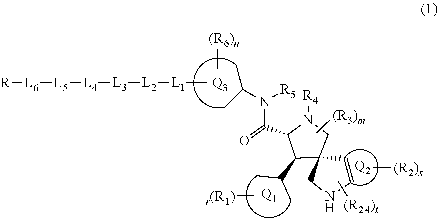

### REFERENCE TO RELATED APPLICATION

This application claims priority to and the benefit of the filing date of U.S. Provisional Patent Application No. 63/345,172, filed on May 24, 2022, the entire contents of which are incorporated herein by reference.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the pI6INK4/pI9ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells.

Phthalimide-based drugs, e.g., thalidomide or lenalidomide, bind to protein-degradation machinery, e.g., cereblon (CRBN; part of an ubiquitin E3 ligase complex). This may promote the recruitment of two transcription factors (IKZF1 and IKZF3) that are essential to disease progression, resulting in drug-induced ubiquitylation and degradation by the proteasome. See, e.g., Ito et al., Science 327: 1345-1350 (2010) and Winter et al., Science 345: 1376-1381 (2015).

A high-affinity VHL ligand, see Bondeson et al., Nat. Chem. Biol. 11:611-617 (2015), may recruit a target protein to an E3 ubiquitin ligase, resulting in drug induced ubiquitination and degradation. See, e.g., van Hagen et al, Nucleic Acids Research 38: 1922-1931 (2010); Buckley et al, J. Am. Chem. Soc. 734:4465-4468 (2012); Buckley et al, Angew, Chem. Int. Ed. Engl. 57: 11463-11467 (2012); Lipkowitz and Weissman, Nat Rev Cancer 11:629-643 (2011); and Zengerle et al, ACS Chem. Biol. 70:1770-1777 (2015).

Although MDM2 inhibitor have made a significant contribution to the art, there is a strong need for continuing search in this field of art for improved pharmaceuticals.

### SUMMARY OF THE INVENTION

The present invention relates to a class of MDM2 inhibitors and degraders. Thus, the compounds of the present invention may be useful in treating the cancer patient. The compounds of the present invention may also be useful in treating patients with diseases such as autoimmune disease, or inflammatory disorders.

In one aspect, this invention relates to a compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof:

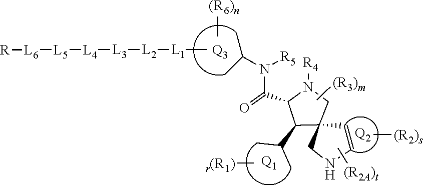

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, (CRₐR_{b})ₚ, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, bivalent alkyl, bivalent alkenyl, bivalent alkynyl, bivalent cycloalkyl, bivalent cycloalkenyl, bivalent heterocycloalkyl, bivalent spirocycloalkyl, bivalent fused-carbocyclic, bivalent bridged-carbocyclic, bivalent heterocycloalkenyl, bivalent spiro-heterocyclic, bivalent fused-heterocyclic, bivalent bridged-heterocyclic, bivalent aryl, or bivalent heteroaryl, each of which is independently optionally substituted with one or more R_{d};
   each of Q₁, Q₂, and Q₃, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
   each of R₁, R₂, R_{2A}, R₃, R₄, R₅, and R₆, independently, is H, D, alkyl, spiroalkyl, alkenyl, alkynyl, halo, nitro, oxo, cyano, —ORₐ, —SRₐ, -alkyl-Rₐ, -alkyl-O—Rₐ, —P(O)(Rₐ)(R_{b}), —P(O)(ORₐ)(OR_{b}), -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
   each Rₐ, R_{b}, R_{c} and Rd, independently, is H, D, alkyl, spiroalkyl, alkenyl, alkynyl, halo, cyano, amine, nitro, hydroxy, ═O, -alkyl-O-Re, -alkyl-O—P(O)(OH)(OH), C(O)NHOH, C(O)OH, —C(O)O—Rₑ, —C(O)—Rₑ, C(O)NH₂, alkoxy, alkoxyalkyl, —O—Rₑ, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, each of which is independently optionally substituted with one or more Rₑ;
   Rₑ is H, D, alkyl, spiroalkyl, alkenyl, alkynyl, halo, cyano, amine, nitro, hydroxy, ═O, -alkyl-O—P(O)(OH)(OH), —C(O)NHOH, —C(O)O—R_{f}, —OC(O)—R_{f}, —C(O)—R_{f}, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, each of which is independently optionally substituted with one or more R_{f}; and
   R_{f} is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
   two of R₁ groups, taken together with the atom to which they are attached, may optionally form a cycloalkyl, heterocycloalkyl, heterocycloalkenyl, aryl, or heteroaryl, each optionally substituted with one or more R_{d};
   two of R₂ groups, taken together with the atom to which they are attached, may optionally form a cycloalkyl, heterocycloalkyl, heterocycloalkenyl, aryl, or heteroaryl, each optionally substituted with one or more R_{d};
   two of R₃ groups, taken together with the atom to which they are attached, may optionally form a cycloalkyl, heterocycloalkyl, heterocycloalkenyl, aryl, or heteroaryl, each optionally substituted with one or more R_{d};
   two of R₆ groups, taken together with the atom to which they are attached, may optionally form a cycloalkyl, heterocycloalkyl, heterocycloalkenyl, aryl, or heteroaryl, each optionally substituted with one or more R_{d};
   R₃ and R₄ groups, taken together with the atom to which they are attached, may optionally form a cycloalkyl, heterocycloalkyl, heterocycloalkenyl, aryl, or heteroaryl, each optionally substituted with one or more R_{d};
   R₄ and R₅ groups, taken together with the atom to which they are attached, may optionally form a cycloalkyl, heterocycloalkyl, heterocycloalkenyl, aryl, or heteroaryl, each optionally substituted with one or more R_{d}; and
   R₅ and R₆ groups, taken together with the atom to which they are attached, may optionally form a cycloalkyl, heterocycloalkyl, heterocycloalkenyl, aryl, or heteroaryl, each optionally substituted with one or more R_{d};
   two of R_{d} groups, taken together with the atom to which they are attached, may optionally form a cycloalkyl, heterocycloalkyl, heterocycloalkenyl, aryl, or heteroaryl, each optionally substituted with one or more Rₑ;
   two of Rₑ groups, taken together with the atom to which they are attached, may optionally form a cycloalkyl, heterocycloalkyl, heterocycloalkenyl, aryl, or heteroaryl, each optionally substituted with one or more R_{f}; and
   each of m, n, r, s, and t, independently, is 0, 1, 2, or 3;
   each of p and q, independently, is 0, 1, 2, 3, or 4.

In one embodiment, the compound is represented by Formula (2):

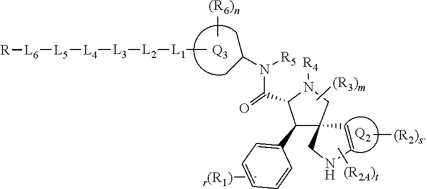

In one embodiment, the compound is represented by Formula (3):

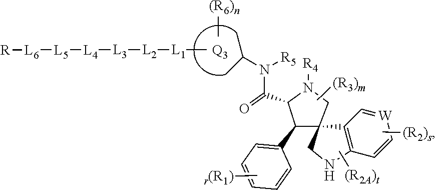

in which W is CH or N.

In preferred embodiments, the compound is represented by Formula (4):

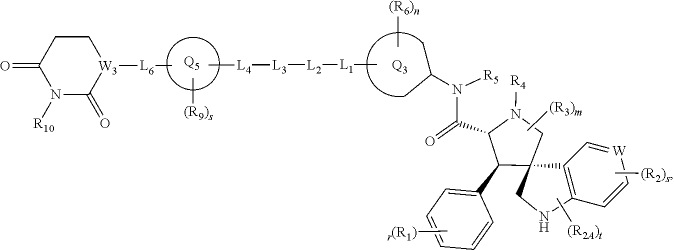

wherein
   R₁₀ is H, D, -alkyl-O—P(O)(Rₐ)(R_{b}), or -alkyl-OC(O)—Rₐ;
   W₃ is N or CH;
   L₆ is absent, NH, CONH, or O;
   Q₅ is absent, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
   R₉ is absent, H, D, alkyl, spiroalkyl, alkenyl, alkynyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, each of which is independently optionally substituted with one or more R_{d};
   R₉ and L₄ groups, taken together with the atom to which they are attached, may optionally form a cycloalkyl, heterocycloalkyl, heterocycloalkenyl, aryl, or heteroaryl, each optionally substituted with one or more R_{d}; and
   s is 0, 1, 2, 3, or 4.

In one embodiment, the compound is represented by Formula (5):

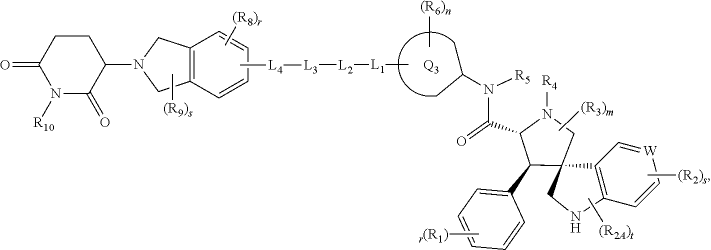

wherein
   R₈ is absent, H, D, alkyl, alkenyl, alkynyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, each of which is independently optionally substituted with one or more R_{d};
   R₈ and L₄ groups, taken together with the atom to which they are attached, may optionally form a cycloalkyl, heterocycloalkyl, heterocycloalkenyl, aryl, or heteroaryl, each optionally substituted with one or more R_{d}; and
   r is 0, 1, 2, 3, or 4;
wherein the remaining groups are as defined in Formula (I).

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-(2-(2,6-dioxopiperidin-3-yl)-1-oxoisoindolin-5-yl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-(2-(2,6-dioxopiperidin-3-yl)-1-oxoisoindolin-5-yl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(7-(2-(2,6-dioxopiperidin-3-yl)-1-oxoisoindolin-5-yl)-2,7-diazaspiro[3.5]nonane-2-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(8-(2-(2,6-dioxopiperidin-3-yl)-1-oxoisoindolin-5-yl)-2,8-diazaspiro[4.5]decane-2-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(9-(2-(2,6-dioxopiperidin-3-yl)-1-oxoisoindolin-5-yl)-3,9-diazaspiro[5.5]undecane-3-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(4-(2-(2,6-dioxopiperidin-3-yl)-1-oxoisoindolin-5-yl)piperazin-1-yl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(2-(4-(2-(2,6-dioxopiperidin-3-yl)-1-oxoisoindolin-5-yl)piperazin-1-yl)-7-azaspiro[3.5]nonane-7-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3aR,6aR)-5-(2-(2,6-dioxopiperidin-3-yl)-1-oxoisoindolin-5-yl)octahydropyrrolo[3,4-c]pyrrole-2-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3aS,6aS)-5-(2-(2,6-dioxopiperidin-3-yl)-1-oxoisoindolin-5-yl)octahydropyrrolo[3,4-c]pyrrole-2-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3aR,6aS)-5-(2-(2,6-dioxopiperidin-3-yl)-1-oxoisoindolin-5-yl)octahydropyrrolo[3,4-c]pyrrole-2-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-((2-(2,6-dioxopiperidin-3-yl)-1-oxoisoindolin-5-yl)methyl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-((2-(2,6-dioxopiperidin-3-yl)-1-oxoisoindolin-5-yl)amino)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-((2-(2,6-dioxopiperidin-3-yl)-1-oxoisoindolin-5-yl)amino)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-((4-(2-(2,6-dioxopiperidin-3-yl)-1-oxoisoindolin-5-yl)piperazin-1-yl)methyl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-isopropoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1,3-dioxoisoindolin-5-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-1-methyl-2-neopentyl-6′-(trifluoromethyl)-1′,2′-dihydrospiro[pyrrolidine-3,3′-pyrrolo[3,2-c]pyridine]-5-carboxamide,
1-((2S,3S,4S,5R)-4-(3-chloro-2-fluorophenyl)-5-((4-(4-(2-((S)-2,6-dioxopiperidin-3-yl)-1-oxoisoindolin-5-yl)piperazine-1-carbonyl)-2-methoxyphenyl)carbamoyl)-1-methyl-2-neopentyl-6′-(trifluoromethyl)spiro[pyrrolidine-3,3′-pyrrolo[3,2-c]pyridin]-1′(2′H)-yl)ethyl isobutyrate,
1-((2S,3S,4S,5R)-4-(3-chloro-2-fluorophenyl)-5-((4-(4-(2-(2,6-dioxo-1-((phosphonooxy)methyl)piperidin-3-yl)-1-oxoisoindolin-5-yl)piperazine-1-carbonyl)-2-methoxyphenyl)carbamoyl)-1-methyl-2-neopentyl-6′-(trifluoromethyl)spiro[pyrrolidine-3,3′-pyrrolo[3,2-c]pyridin]-1′(2′H)-yl)ethyl isobutyrate,
(2S,3S,4S,5R)-4-(3-chloro-2-fluorophenyl)-2-cyclopropyl-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-2-cyclobutyl-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-2-((S)-2,2-dimethylcyclobutyl)-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-2-cyclopentyl-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-2-cyclohexyl-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-2-((R)-2,2-dimethylcyclohexyl)-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-2-((S)-2,2-dimethylcyclohexyl)-N-(4-(4-(2-((S)-2,6-dioxopiperidin-3-yl)-1-oxoisoindolin-5-yl)piperazine-1-carbonyl)-2-methoxyphenyl)-1-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-1-methyl-2-(7-oxaspiro[3.5]nonan-2-yl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-1-methyl-2-(3-morpholinocyclobutyl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-1,2-dimethyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-2-methyl-1-(tetrahydro-2H-pyran-4-yl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-2-methyl-1-(7-oxaspiro[3.5]nonan-2-yl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-1-(4-fluorophenyl)-2-methyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-2-methyl-1-(pyridin-4-yl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-2-methyl-1-((tetrahydro-2H-pyran-4-yl)methyl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-2-methyl-1-((tetrahydro-2H-pyran-4-yl)methyl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-((S)-2,6-dioxopiperidin-3-yl)-1-oxoisoindolin-5-yl)piperazine-1-carbonyl)-2-methoxyphenyl)-2-methyl-1-(pyridin-3-ylmethyl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-(methoxy-d3)phenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)phenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methylphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-(trifluoromethoxy)phenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-N-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-4-(2-(2,6-dioxopiperidin-3-yl)-1-oxoisoindolin-5-yl)-3-methylpiperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-4-(2-(2,6-dioxopiperidin-3-yl)-1-oxoisoindolin-5-yl)-3-methylpiperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((2R)-4-(2-(2,6-dioxopiperidin-3-yl)-1-oxoisoindolin-5-yl)-2-methylpiperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((2S)-4-(2-(2,6-dioxopiperidin-3-yl)-1-oxoisoindolin-5-yl)-2-methylpiperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-((tetrahydro-2H-pyran-4-yl)oxy)phenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(1-(2-(2,6-dioxopiperidin-3-yl)-1-oxoisoindolin-5-yl)piperidin-4-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((R)-2-(2-((S)-2,6-dioxopiperidin-3-yl)-1-oxoisoindolin-5-yl)morpholine-4-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((S)-2-(2-((S)-2,6-dioxopiperidin-3-yl)-1-oxoisoindolin-5-yl)morpholine-4-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-((2-(2,6-dioxopiperidin-3-yl)-1-oxoisoindolin-5-yl)oxy)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-((2-(2,6-dioxopiperidin-3-yl)-1-oxoisoindolin-5-yl)oxy)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-((2-(2,6-dioxopiperidin-3-yl)-1-oxoisoindolin-5-yl)amino)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-((2-(2,6-dioxopiperidin-3-yl)-1-oxoisoindolin-5-yl)amino)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-((2-(2,6-dioxopiperidin-3-yl)-1-oxoisoindolin-5-yl)oxy)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-((2-(2,6-dioxopiperidin-3-yl)-1-oxoisoindolin-5-yl)amino)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-2-methyl-1-(tetrahydro-2H-pyran-4-yl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-1-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-1-benzyl-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-2-neopentyl-1-(pyridin-2-yl)-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-1-isopropyl-2-neopentyl-6′-(trifluoromethyl)-1′,2′-dihydrospiro[pyrrolidine-3,3′-pyrrolo[3,2-c]pyridine]-5-carboxamide,
1-(3-(5-(4-(4-((2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxamido)-3-methoxybenzoyl)piperazin-1-yl)-1-oxoisoindolin-2-yl)-2,6-dioxopiperidin-1-yl)ethyl dihydrogen phosphate,
1-((S)-3-(5-((S)-4-(4-((2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxamido)-3-methoxybenzoyl)-2-methylpiperazin-1-yl)-1-oxoisoindolin-2-yl)-2,6-dioxopiperidin-1-yl)ethyl dihydrogen phosphate,
1-(isobutyryloxy)ethyl (2S,3S,4S,5R)-4-(3-chloro-2-fluorophenyl)-5-((4-(4-(2-(2,6-dioxopiperidin-3-yl)-1-oxoisoindolin-5-yl)piperazine-1-carbonyl)-2-methoxyphenyl)carbamoyl)-1-methyl-2-neopentyl-6′-(trifluoromethyl)spiro[pyrrolidine-3,3′-pyrrolo[3,2-c]pyridine]-1′(2′H)-carboxylate,
1-((2S,3S,4S,5R)-4-(3-chloro-2-fluorophenyl)-5-((4-(4-(2-(2,6-dioxopiperidin-3-yl)-1-oxoisoindolin-5-yl)piperazine-1-carbonyl)-2-methoxyphenyl)carbamoyl)-1-methyl-2-neopentyl-6′-(trifluoromethyl)spiro[pyrrolidine-3,3′-pyrrolo[3,2-c]pyridin]-1′(2′H)-yl)ethyl isobutyrate,
((2S,3S,4S,5R)-4-(3-chloro-2-fluorophenyl)-5-((4-(4-(2-(2,6-dioxopiperidin-3-yl)-1-oxoisoindolin-5-yl)piperazine-1-carbonyl)-2-methoxyphenyl)carbamoyl)-1-methyl-2-neopentyl-6′-(trifluoromethyl)spiro[pyrrolidine-3,3′-pyrrolo[3,2-c]pyridin]-1′(2′H)-yl)methyl dihydrogen phosphate,
((2S,3S,4S,5R)-4-(3-chloro-2-fluorophenyl)-5-((4-(4-(2-(2,6-dioxopiperidin-3-yl)-1-oxoisoindolin-5-yl)piperazine-1-carbonyl)-2-methoxyphenyl)carbamoyl)-1-methyl-2-neopentyl-6′-(trifluoromethyl)spiro[pyrrolidine-3,3′-pyrrolo[3,2-c]pyridin]-1′(2′H)-yl)phosphonic acid,
(2S,3S,4S,5R)-4-(3-chloro-2-fluorophenyl)-N-(4-(4-(7-(2-(2,6-dioxopiperidin-3-yl)-1-oxoisoindolin-5-yl)-7-azaspiro[3.5]nonan-2-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(3-(2-(2,6-dioxopiperidin-3-yl)-1-oxoisoindolin-5-yl)azetidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-(2-(2,6-dioxopiperidin-3-yl)-1-oxoisoindolin-5-yl)pyrrolidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-(2-(2,6-dioxopiperidin-3-yl)-1-oxoisoindolin-5-yl)pyrrolidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)-1,4-diazepane-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(2-(2-(2,6-dioxopiperidin-3-yl)-1-oxoisoindolin-5-yl)-2,7-diazaspiro[3.5]nonane-7-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide, (3'S,4'S,5′R)-6″-chloro-4′-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)dispiro[cyclobutane-1,2′-pyrrolidine-3′,3″-indoline]-5′-carboxamide,
(3'S,4'S,5′R)-4′-(3-chloro-2-fluorophenyl)-N-(4-(4-(2-(2,6-dioxopiperidin-3-yl)-1-oxoisoindolin-5-yl)piperazine-1-carbonyl)-2-methoxyphenyl)-3,3-dimethyl-6″-(trifluoromethyl)-1″,2″-dihydrodispiro[cyclobutane-1,2′-pyrrolidine-3′,3″-pyrrolo[3,2-c]pyridine]-5′-carboxamide,
(2S,3S,4S,5R)-4-(3-chloro-2-fluorophenyl)-N-(4-(3-(2-(2,6-dioxopiperidin-3-yl)-1-oxoisoindolin-5-yl)azetidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-(2-(2,6-dioxopiperidin-3-yl)-1-oxoisoindolin-5-yl)pyrrolidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-(2-(2,6-dioxopiperidin-3-yl)-1-oxoisoindolin-5-yl)pyrrolidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-(2-(2,6-dioxopiperidin-3-yl)-1-oxoisoindolin-5-yl)piperidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-(2-(2,6-dioxopiperidin-3-yl)-1-oxoisoindolin-5-yl)piperidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)-1,4-diazepane-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(2-(2-(2,6-dioxopiperidin-3-yl)-1-oxoisoindolin-5-yl)-2,7-diazaspiro[3.5]nonane-7-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(7-(2-(2,6-dioxopiperidin-3-yl)-1-oxoisoindolin-5-yl)-2,7-diazaspiro[3.5]nonane-2-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(8-(2-(2,6-dioxopiperidin-3-yl)-1-oxoisoindolin-5-yl)-2,8-diazaspiro[4.5]decane-2-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(9-(2-(2,6-dioxopiperidin-3-yl)-1-oxoisoindolin-5-yl)-3,9-diazaspiro[5.5]undecane-3-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3aR,6aR)-5-(2-(2,6-dioxopiperidin-3-yl)-1-oxoisoindolin-5-yl)octahydropyrrolo[3,4-c]pyrrole-2-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3aR,6aS)-5-(2-(2,6-dioxopiperidin-3-yl)-1-oxoisoindolin-5-yl)octahydropyrrolo[3,4-c]pyrrole-2-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3aS,6aS)-5-(2-(2,6-dioxopiperidin-3-yl)-1-oxoisoindolin-5-yl)octahydropyrrolo[3,4-c]pyrrole-2-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-((2-(2,6-dioxopiperidin-3-yl)-1-oxoisoindolin-5-yl)methyl)piperazine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3S)-3-((2-(2,6-dioxopiperidin-3-yl)-1-oxoisoindolin-5-yl)amino)piperidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((3R)-3-((2-(2,6-dioxopiperidin-3-yl)-1-oxoisoindolin-5-yl)amino)piperidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(1-(2-(2,6-dioxopiperidin-3-yl)-1-oxoisoindolin-5-yl)piperidin-4-yl)piperazine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(4-(2-(2,6-dioxopiperidin-3-yl)-1-oxoisoindolin-5-yl)piperazin-1-yl)piperidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-((4-(2-(2,6-dioxopiperidin-3-yl)-1-oxoisoindolin-5-yl)piperazin-1-yl)methyl)piperidine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(2-(4-(2-(2,6-dioxopiperidin-3-yl)-1-oxoisoindolin-5-yl)piperazin-1-yl)-7-azaspiro[3.5]nonane-7-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(7-(2-(2,6-dioxopiperidin-3-yl)-1-oxoisoindolin-5-yl)-7-azaspiro[3.5]nonan-2-yl)piperazine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-N-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-N-isopropyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-cyclopropyl-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-1-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-N,1-dimethyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-N-isopropyl-1-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-cyclopropyl-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-1-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-1-ethyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-1-ethyl-N-methyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-1-ethyl-N-isopropyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-cyclopropyl-N-(4-((1S)-1-((2-(2,6-dioxopiperidin-3-yl)-1-oxoisoindolin-4-yl)ethynyl)-6-azaspiro[2.5]octane-6-carbonyl)bicyclo[2.2.2]octan-1-yl)-1-ethyl-2-neopentyl-6′-(trifluoromethyl)-1′,2′-dihydrospiro[pyrrolidine-3,3′-pyrrolo[3,2-c]pyridine]-5-carboxamide,
(2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)bicyclo[2.2.2]octan-1-yl)-2-neopentyl-6′-(trifluoromethyl)-1′,2′-dihydrospiro[pyrrolidine-3,3′-pyrrolo[3,2-c]pyridine]-5-carboxamide

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-a-tocopheryl PEG 1000 succinate (20-50%), using complexation such as HPRCD and SBERCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl.

The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

Spirocycloalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalikyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups:

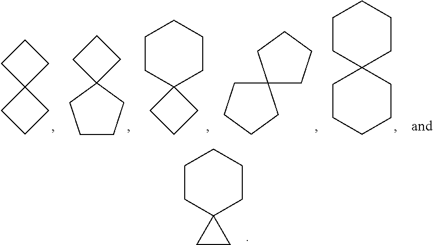

The term “fused-carbocyclic” refers to a polycyclic cyclyl group, wherein each ring in the group shares an adjacent pair of carbon atoms with another ring in the group, wherein one or more rings can contain one or more double bonds. In certain embodiments, the fused heterocyclyl is bicyclic. In certain embodiments, the fused-carbocyclic contains more than two rings, at least two of which share an adjacent pair of atoms. In one embodiment, the fused-carbocyclic is a 5 to 20 membered, 5 to 16 membered, or 5 to 10 membered polycyclic cyclyl group. Representative examples of fused-carbocyclic include, but are not limited to the following groups:

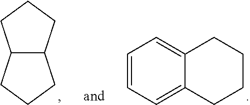 [SMILES: C1CC2CCCC2C1 ; c1ccc2c(c1)CCCC2]

The term “bridged-carbocyclic” refers to a group having at least two rings sharing three or more common ring atoms, separating the two bridgehead atoms by a bridge containing at least one atom. The bridgehead atoms are the atoms from which three bonds radiate and where the rings meet. The rings of the bridged carbocyclyl can have one or more double bonds. In one embodiment, the bridged carbocyclyl is bicyclic. In one embodiment, the bridged carbocyclyl is a 5 to 20 membered, 5 to 16 membered, or 5 to 10 membered polycyclic carbocyclyl group. Representative examples of bridged carbocyclyl include, but are not limited to the following groups:

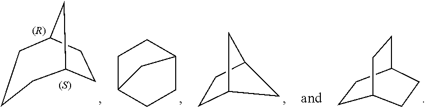

The term “heterocycloalkyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se) and one or more double bonds.

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

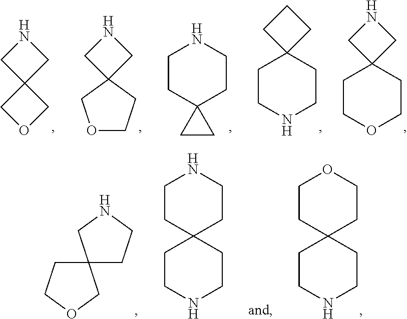

Fused heterocyclyl refers to a polycyclic heterocyclyl group, wherein each ring in the group shares an adjacent pair of atoms (such as carbon atoms) with another ring in the group, wherein one or more rings can contain one or more double bonds, and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic. In certain embodiments, the fused heterocyclyl contains more than two rings, at least two of which share an adjacent pair of atoms. In one embodiment, the fused heterocyclyl is a 5 to 20 membered, 5 to 16 membered, or 5 to 10 membered polycyclic heterocyclyl group. Representative examples of fused heterocyclyl include, but are not limited to the following groups:

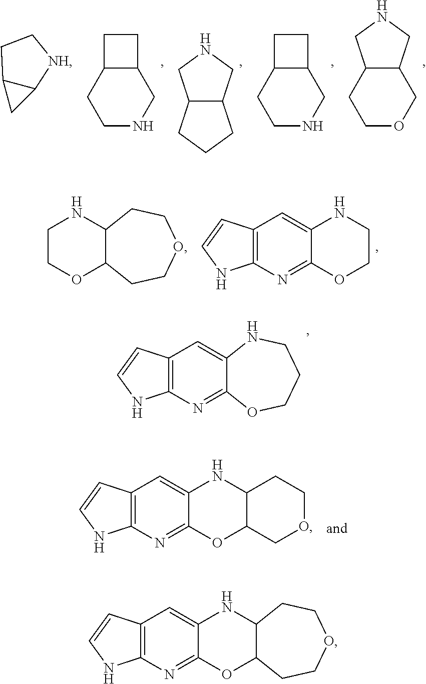

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 16 membered, or 5 to 10 membered polycyclic heterocyclyl group. Representative examples of bridged heterocyclyl include, but are not limited to the following groups:

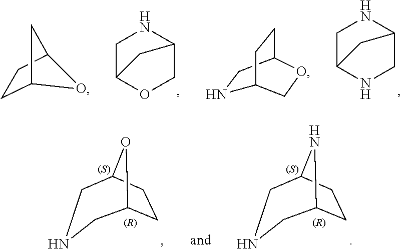

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, mentioned above include both substituted and unsubstituted moieties. Possible substituents on alkyl, alkenyl, alkynyl, alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroarylinclude, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T.W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-R-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIla, CAMKIIb, CAMKIld, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin Al, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4NEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kbl, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q252H), ABL1(T3151), ABL1(Y253F), ALK (C1156Y), ALK(L1196M), ALK (F1174L), ALK (R1275Q), BRAF(V599E), BTK(E41K), CHK2(1157T), c-Kit(A829P), c-KIT(D816H), c-KIT(D816V), c-Kit(D820E), c-Kit(N822K), C-Kit (T6701), c-Kit(V559D), c-Kit(V559D/V654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (I2020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARP inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barre syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

An approach to synthesize the intermediates

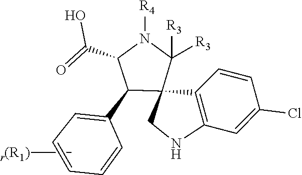

is described in Scheme A. R₁, R₃, R₄, and r, in general Scheme A is the same as those described in the Summary section above.

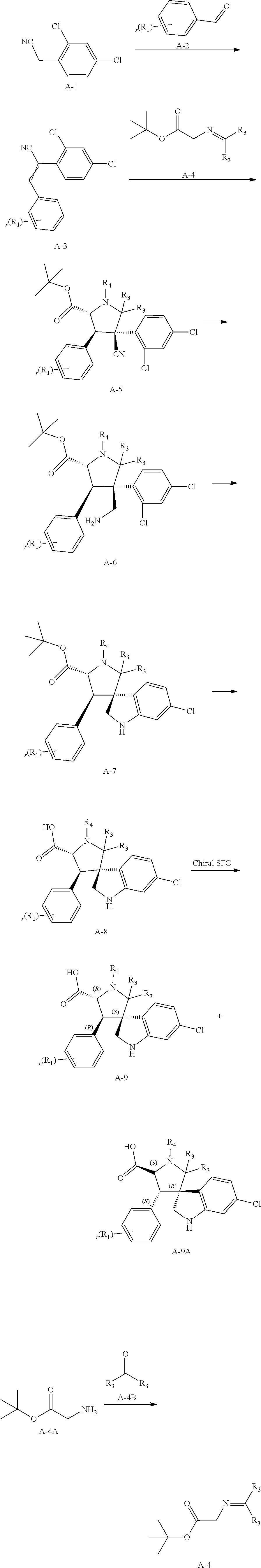

In Scheme A, the starting material A-1 can be prepared by conventional procedures using appropriate compounds and reagents. A-1 can react with A-2 to afford A-3, which can undergo an intermolecular [3+2]cyclization with A-4 derived from A-4A, to give A-5. After that, A-5 can be reduced to A-6, and then A-6 can go through an intramolecular coupling reaction to give A-7. A-7 can be transformed to A-8. Finally, A-9 can be obtained from A-8 by chiral separation.

Also, the target compounds can be synthesized by alternative methods but not limited to the above procedures.

An approach to synthesize the intermediates

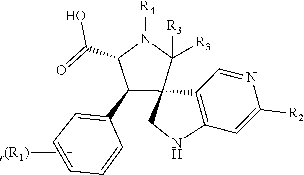

is described in Scheme B. R₁, R₂, R₃, R₄, and r, in general Scheme B is the same as those described in the Summary section above.

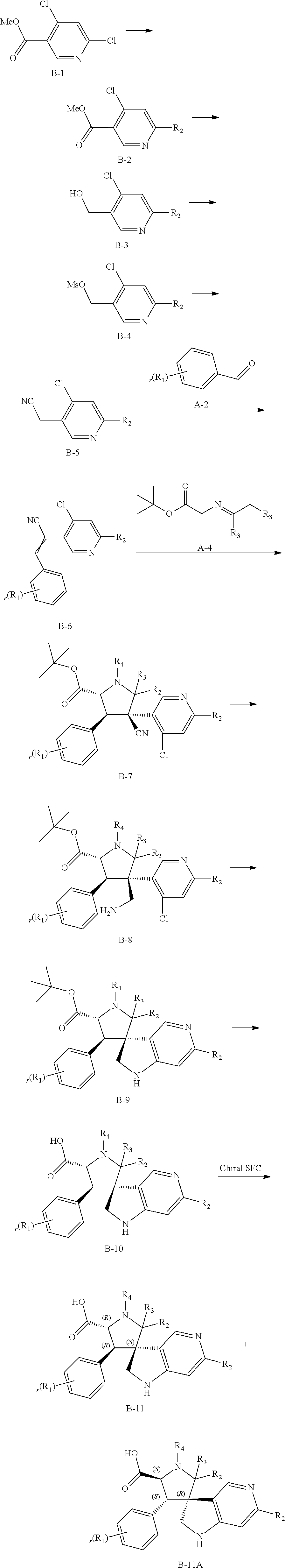

In Scheme B, the starting material B-1 can be prepared by conventional procedures using appropriate compounds and reagents. The starting material B-1 is converted to B-2 by a coupling reaction, which can further be reduced to the alcohol intermediate B-3. After that, B-3 can go through a standard condition to yield B-4, which can subsequently be converted to B-5. Next, the cyanide B-5 can react with A-2 to afford B-6, which can under an intermolecular [3+2]cyclization with A-4 to give B-7. Next, B-7 is reduced to yield B-8, and the subsequent intramolecular coupling reaction to give B-9. B-9 can be hydrolyzed to B-10 under a suitable acid condition. Finally, B-11 can be obtained from B-10 by chiral separation.

Also, the target compounds can be synthesized by alternative methods but not limited to the above procedures.

The intermediate

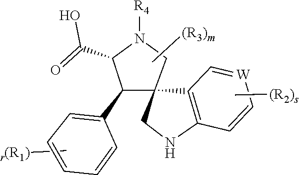

can be made by the method similar to Scheme A and B, by using different starting material and reagents, or by the standard organic reactions.

The intermediate

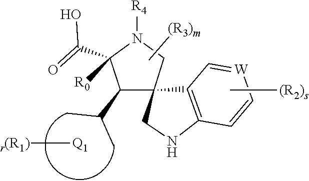

can be made by the method similar to Scheme A and B, by using different starting material and reagents, or by the standard organic reactions.

The intermediate

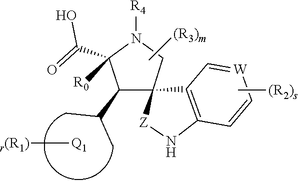

can be made by the method similar to Scheme A and B, by using different starting material and reagents, or by the standard organic reactions.

An approach to synthesize compounds of

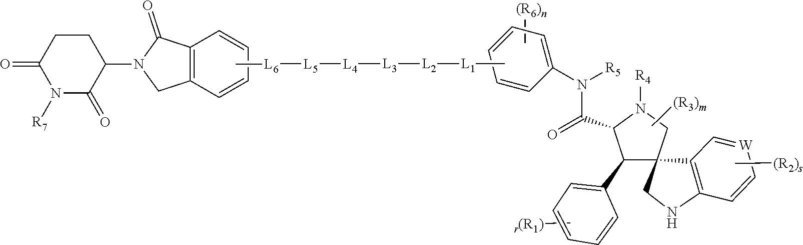

is described in Scheme 1. A, W, R₁, R₂, R₃, R₄, R₅, R₆, R₇, L₁, L₂, L₃, L₄, L₅, L₆, r, s, n, and m, in general Scheme 1 is the same as those described in the Summary section above.

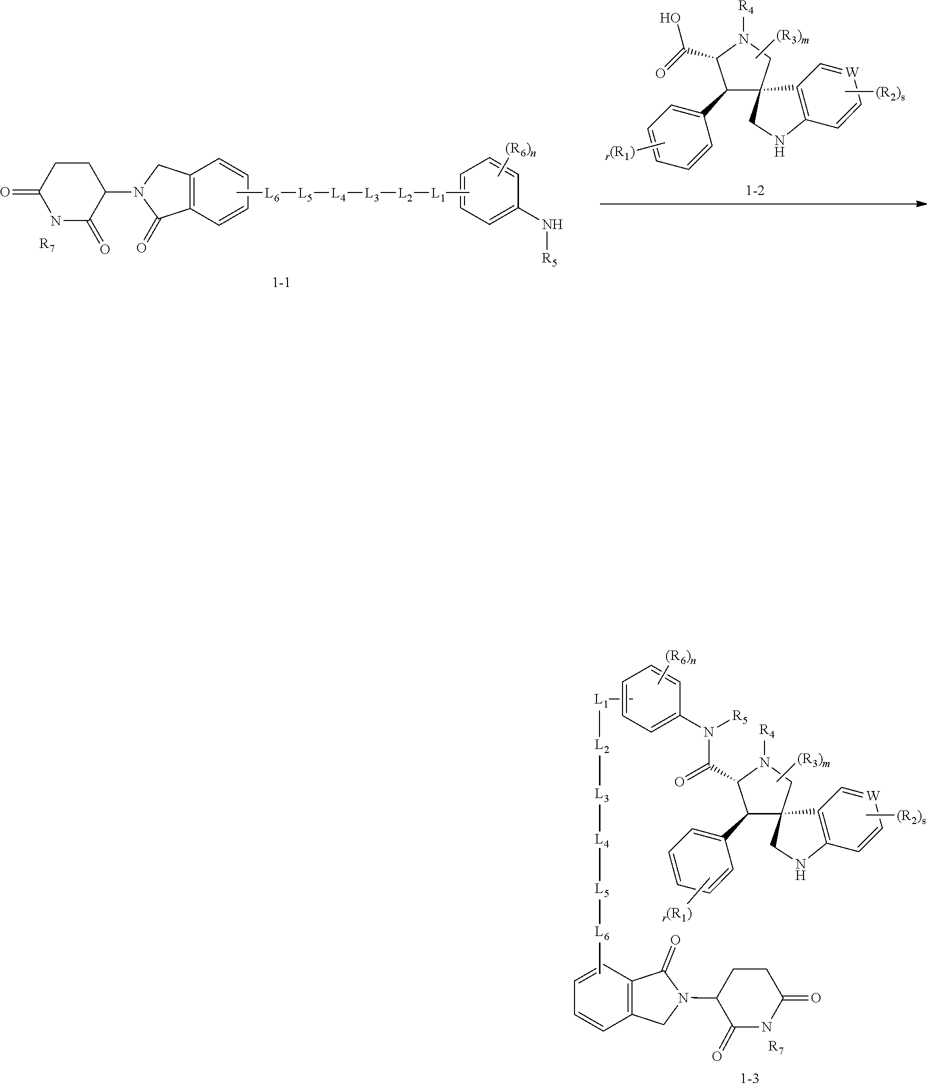

In Scheme 1, the starting material 1-1 can be prepared by standard organic reaction. The intermediate 1-2 can be prepared by the method similar to Scheme A and B, by using different starting material and reagents, or by the standard organic reactions. The amide coupling of 1-1 and 1-2 can afford the target compound 1-3.

The compounds

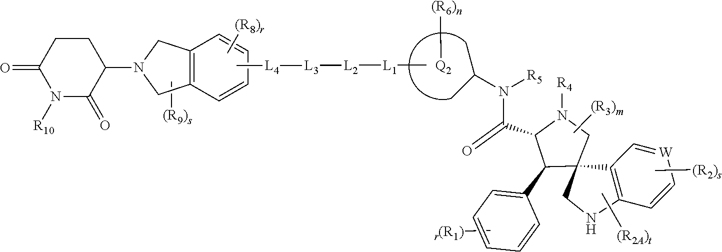

can be made by the method similar to Scheme 1, by using different starting material and reagents, or by the standard organic reactions.

The compounds

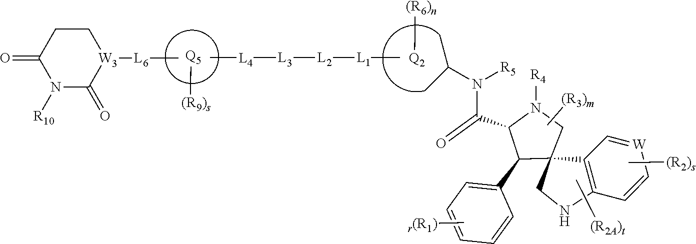

can be made by the method similar to Scheme 1, by using different starting material and reagents, or by the standard organic reactions.

The compounds

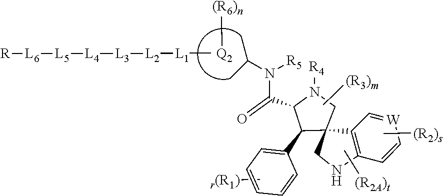

can be made by the method similar to Scheme 1, by using different starting material and reagents, or by the standard organic reactions.

The compounds

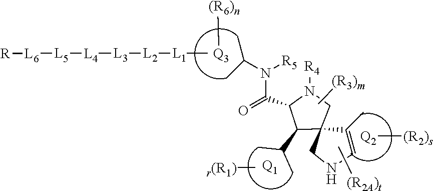

can be made by the method similar to Scheme 1, by using different starting material and reagents, or by the standard organic reactions.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example INT-1: Preparation of tert-butyl 2-((3,3-dimethylbutylidene)amino)acetate

### Synthesis of tert-butyl 2-((3,3-dimethylbutylidene)amino)acetate

Into a 2000-mL 3-necked round-bottom flask were placed tert-butyl 2-aminoacetate (70.0 g, 533.6 mmol, 1.0 eq), dichloromethane (700 mL), 3,3-dimethylbutanal (56.1 g, 560.3 mmol, 1.0 eq). The reaction mixture was stirred overnight at 25° C. The resulting mixture was concentrated under vacuum to give tert-butyl 2-((3,3-dimethylbutylidene)amino)acetate (135.0 g, crude) as light yellow oil. ¹HNMR (300 MHz, DMSO-d₆) δ 7.71 (t, J=5.7 Hz, 1H), 4.04 (s, 2H), 2.10 (d, J=5.6 Hz, 2H), 1.41 (s, 9H), 0.95 (s, 9H).

### Example INT-2: Preparation of 3-(3-chloro-2-fluorophenyl)-2-(2,4-dichlorophenyl) acrylonitrile

### Synthesis of 3-(3-chloro-2-fluorophenyl)-2-(2,4-dichlorophenyl) acrylonitrile

Into a 2000-mL 3-necked round-bottom flask were placed 2-(2,4-dichlorophenyl) acetonitrile (70.0 g, 376.3 mmol, 1.0 eq), methanol (700 mL), 3-chloro-2-fluorobenzaldehyde (59.7 g, 376.3 mmol, 1.0 eq). After that, sodium methanolate (30.5 g, 564.4 mmol, 1.5 eq) was added at 0° C. The reaction mixture was stirred for 3 hours at 50° C. The precipitated solids were collected by filtration and washed with methanol (2×300 mL). Finally, 3-(3-chloro-2-fluorophenyl)-2-(2,4-dichlorophenyl) acrylonitrile (110.0 g, 89%) was obtained as a light yellow solid. ¹HNMR (400 MHz, Chloroform-d) δ 8.21 (ddd, J=8.0, 6.4, 1.8 Hz, 1H), 7.59-7.51 (m, 3H), 7.43 (d, J=8.4 Hz, 1H), 7.38 (dd, J=8.4, 2.2 Hz, 1H), 7.31-7.21 (m, 1H).

### Example INT-3 (Method A): Preparation of (2'S,3S,4'S,5′R)-6-chloro-4′-(3-chloro-2-fluorophenyl)-2′-neopentylspiro[indoline-3,3′-pyrrolidine]-5′-carboxylic acid

### Synthesis of tert-butyl (2R,3S,4R,5S)-3-(3-chloro-2-fluorophenyl)-4-cyano-4-(2,4-dichlorophenyl)-5-neopentylpyrrolidine-2-carboxylate (racemate)

Into a 2000-mL 3-necked round-bottom flask were placed 3-(3-chloro-2-fluorophenyl)-2-(2,4-dichlorophenyl) acrylonitrile (100.0 g, 306.2 mmol, 1.0 eq), tert-butyl 2-((3,3-dimethylbutylidene)amino)acetate (117.6 g, 551.2 mmol, 1.8 eq), 1,2-dichloroethane (1000 mL), AgF (46.6 g, 367.4 mmol, 1.2 eq), triethylamine (61.9 g, 612.4 mmol, 2.0 eq). The reaction mixture was stirred for 16 hours at 25° C. The resulting mixture was filtered and the filtrate was concentrated under vacuum. The crude residue was applied onto a silica gel column and eluted with ethyl acetate/petroleum ether=(0:1 to 1:3) to give tert-butyl (2R,3S,4R,5S and 2S,3R,4S,5R)-3-(3-chloro-2-fluorophenyl)-4-cyano-4-(2,4-dichlorophenyl)-5-neopentylpyrrolidine-2-carboxylate (racemate) (76.0 g, 46%) as a light yellow solid. LC-MS (ESI, m/z) M+1: 539/541. ¹HNMR (300 MHz, Methanol-d₄) δ 7.75 (d, J=6.6 Hz, 1H), 7.70 (d, J=2.1 Hz, 1H), 7.56 (d, J=8.1 Hz, 1H), 7.51-7.44 (m, 1H), 7.42 (dd, J=8.4, 2.4 Hz, 1H), 7.28 (t, J=8.1 Hz, 1H), 5.35 (d, J=9.3 Hz, 1H), 5.0 (dd, J=8.7, 1.4 Hz, 2H), 4.80 (d, J=9.3 Hz, 1H), 1.89 (dd, J=15.0, 8.7 Hz, 1H), 1.48 (d, J=13.5 Hz, 1H), 1.38 (s, 9H), 0.95 (s, 9H).

### Synthesis of tert-butyl (2R,3S,4S,5S and 2S,3R,4R,5R)-4-(aminomethyl)-3-(3-chloro-2-fluorophenyl)-4-(2,4-dichlorophenyl)-5-neopentylpyrrolidine-2-carboxylate (racemate)

Into a 2000-mL round-bottom flask were placed tert-butyl (2R,3S,4R,5S)-3-(3-chloro-2-fluorophenyl)-4-cyano-4-(2,4-dichlorophenyl)-5-neopentylpyrrolidine-2-carboxylate (racemate) (28.0 g, 51.9 mmol, 1.0 eq), methanol (500 mL), acetic acid (125 mL), water (125 mL) and Raney-Ni (5.0 g). The reaction mixture was stirred at 50° C. under hydrogen (2 atm) for 48 hours. The resulting mixture was filtered, and the filtrate was concentrated under vacuum. Finally, tert-butyl (2R,3S,4S,5S and 2S,3R,4R,5R)-4-(aminomethyl)-3-(3-chloro-2-fluorophenyl)-4-(2,4-dichlorophenyl)-5-neopentylpyrrolidine-2-carboxylate (racemate) (20.0 g, 71%) was obtained as an off white solid. LC-MS (ESI, m/z) M+1: 543/545. ¹HNMR (300 MHz, DMSO-d₆) δ 8.02 (s, 3H), 7.62 (d, J=2.4 Hz, 1H), 7.56-7.52 (m, 3H), 7.32 (d, J=8.4 Hz, 1H), 7.27 (t, J=7.8 Hz, 1H), 4.52 (d, J=12.0 Hz, 2H), 4.16 (d, J=8.4 Hz, 1H), 3.60-3.43 (m, 2H), 1.47 (dd, J=14.1, 10.2 Hz, 1H), 1.40-1.31 (m, 1H), 1.26 (s, 9H), 1.0 (s, 9H).

### Synthesis of tert-butyl (2'S,3S,4'S,5′R)-6-chloro-4′-(3-chloro-2-fluorophenyl)-2′-neopentylspiro[indoline-3,3′-pyrrolidine]-5′-carboxylate (racemate)

Into a 500 mL round-bottom flask and maintained under an inert atmosphere of nitrogen, were placed tert-butyl (2R,3S,4S,5S and 2S,3R,4R,5R)-4-(aminomethyl)-3-(3-chloro-2-fluorophenyl)-4-(2,4-dichlorophenyl)-5-neopentylpyrrolidine-2-carboxylate (racemate) (12.0 g, 22.1 mmol, 1.0 eq), methyl sulfoxide (240 mL), K₃PO₄ (18.7 g, 88.2 mmol, 4.0 eq), CuI (0.8 g, 13.2 mmol, 0.6 eq) and (1S,2S)-N,N′-dimethyl-1,2-diaminocyclohexane (1.3 g, 8.8 mmol, 0.4 eq). The reaction mixture was stirred for 2 hours at 100° C. The resulting mixture was diluted with water (300 mL) to give a suspension. After filtration, the filter cake was collected. The crude residue was applied onto a silica gel column and eluted with ethyl acetate/petroleum ether=(0:1 to 1:1) to give tert-butyl (2'S,3S,4'S,5′R)-6-chloro-4′-(3-chloro-2-fluorophenyl)-2′-neopentylspiro[indoline-3,3′-pyrrolidine]-5′-carboxylate (racemate) (6.0 g, 54%) as light yellow oil. LC-MS (ESI, m/z) M+1: 507/509. ¹HNMR (400 MHz, Methanol-d₄) δ 7.36-7.31 (m, 1H), 7.28 (d, J=6.8 Hz, 1H), 7.12 (d, J=8.0 Hz, 2H), 6.65 (d, J=8.0 Hz, 1H), 6.41 (s, 1H), 4.12 (d, J=9.6 Hz, 1H), 3.99 (d, J=9.6 Hz, 1H), 3.60 (d, J=10.4 Hz, 1H), 3.50 (d, J=7.6 Hz, 1H), 3.47 (d, J=10.4 Hz, 1H), 1.41 (d, J=10.0 Hz, 2H), 1.33 (s, 9H), 0.88 (s, 9H).

### Synthesis of (2'S,3S,4'S,5′R)-6-chloro-4′-(3-chloro-2-fluorophenyl)-2′-neopentylspiro[indoline-3,3′-pyrrolidine]-5′-carboxylic acid (racemate)

Into a 250 mL round-bottom flask were placed tert-butyl (2'S,3S,4'S,5′R)-6-chloro-4′-(3-chloro-2-fluorophenyl)-2′-neopentylspiro[indoline-3,3′-pyrrolidine]-5′-carboxylate (racemate) (5.5 g, 10.8 mmol, 1.0 eq), trifluoroacetic acid (60 mL). The reaction mixture was stirred for 16 hours at 25° C. The resulting mixture was diluted with water (200 mL) and then extracted with ethyl acetate (2×100 mL). The combined organic phase was washed with brine (2×100 mL), dried over anhydrous sodium sulfate. After filtration, the filtrate was concentrated under vacuum. Finally, (2'S,3S,4'S,5′R)-6-chloro-4′-(3-chloro-2-fluorophenyl)-2′-neopentylspiro[indoline-3,3′-pyrrolidine]-5′-carboxylic acid (racemate) (4.7 g, crude) was obtained as light yellow solid. LC-MS (ESI, m/z) M+1: 451/453.

### Example INT-3 (Method B): Preparation of (2'S,3S,4'S,5′R)-6-chloro-4′-(3-chloro-2-fluorophenyl)-2′-neopentylspiro[indoline-3,3′-pyrrolidine]-5′-carboxylic acid

### Synthesis of 2-(2-bromo-4-chlorophenyl)-3-(3-chloro-2-fluorophenyl) acrylonitrile

Into a 250-mL 3-necked round-bottom flask, were placed 2-(2-bromo-4-chlorophenyl)acetonitrile (10.0 g, 43.4 mmol, 1.0 eq), CH₃OH (100 mL), 3-chloro-2-fluorobenzaldehyde (6.9 g, 43.4 mmol, 1.0 eq). After that, MeONa (3.5 g, 65.1 mmol, 1.5 eq) was added at 0° C. The reaction mixture was stirred for 3 hours at 50° C. The precipitated solids were collected by filtration and then washed with CH₃OH (50 mL×2). Finally, 2-(2-bromo-4-chlorophenyl)-3-(3-chloro-2-fluorophenyl) acrylonitrile (13.0 g, 80%) was obtained as a light yellow oil. LC-MS (ESI, m/z) M+1: 370/372. ¹HNMR (300 MHz, Chloroform-d) δ 8.04-7.94 (m, 2H), 7.88-7.72 (m, 1H), 7.72-7.61 (m, 3H), 7.41 (dt, J=31.2, 8.1 Hz, 1H).

### Synthesis of tert-butyl 4-(2-bromo-4-chlorophenyl)-3-(3-chloro-2-fluorophenyl)-4-cyano-5-neopentylpyrrolidine-2-carboxylate

Into a 250-mL 3-necked round-bottom flask, were placed 2-(2-bromo-4-chlorophenyl)-3-(3-chloro-2-fluorophenyl) acrylonitrile (9.0 g, 24.4 mmol, 1.0 eq), tert-butyl 2-((3,3-dimethylbutylidene)amino)acetate (6.2 g, 29.3 mmol, 1.2 eq), 1,2-dichloroethane (90 mL), AgF (3.7 g, 29.3 mmol, 1.2 eq), triethylamine (4.9 g, 48.8 mmol, 2.0 eq). The reaction mixture was stirred for 16 hours at 25° C. The resulting mixture was filtered, the filtrate was concentrated under vacuum. The crude residue was applied onto a silica gel column and eluted with ethyl acetate/petroleum ether=(0:1 to 1:1). Finally, tert-butyl 4-(2-bromo-4-chlorophenyl)-3-(3-chloro-2-fluorophenyl)-4-cyano-5-neopentylpyrrolidine-2-carboxylate (5.0 g, crude) was obtained as an off white solid, which was directly used to the next step.

LC-MS (ESI, m/z) M+1: 583/585.

### Synthesis of tert-butyl (2R, 3S, 4R, 5S)-4-(2-bromo-4-chlorophenyl)-3-(3-chloro-2-fluorophenyl)-4-cyano-5-neopentylpyrrolidine-2-carboxylate(racemate)

Into a 250-mL 3-necked round-bottom flask, were placed tert-butyl 4-(2-bromo-4-chlorophenyl)-3-(3-chloro-2-fluorophenyl)-4-cyano-5-neopentylpyrrolidine-2-carboxylate (crude product, 5.0 g, 1.0 eq), tetrahydrofuran (60 mL), LiOH (0.6 g, 25.7 mmol, 3.0 eq). The reaction mixture was stirred for 14 hours at 70° C. The resulting mixture was filtered, the filtrate was concentrated under vacuum. Finally, tert-butyl (2R, 3S, 4R, 5S)-4-(2-bromo-4-chlorophenyl)-3-(3-chloro-2-fluorophenyl)-4-cyano-5-neopentylpyrrolidine-2-carboxylate (racemate) (4.8 g, crude) was obtained as an off white solid. LC-MS (ESI, m/z) M+1: 583/585. ¹HNMR (300 MHz, DMSO-d₆) δ 7.96 (d, J=2.4 Hz, 1H), 7.70 (t, J=6.6 Hz, 1H), 7.57-7.49 (m, 2H), 7.53-7.43 (m, 1H), 7.37-7.24 (m, 1H), 5.25 (d, J=7.5 Hz, 1H), 4.68 (d, J=9.0 Hz, 1H), 4.34 (d, J=7.5 Hz, 1H), 1.52 (dd, J=14.1, 9.3 Hz, 1H), 1.32 (s, 9H), 1.24-1.16 (m, 1H), 0.89 (s, 9H).

### Synthesis of tert-butyl (2R, 3S, 4R, 5S)-4-(aminomethyl)-4-(2-bromo-4-chlorophenyl)-3-(3-chloro-2-fluorophenyl)-5-neopentylpyrrolidine-2-carboxylate (racemate)

Into a 250-mL 3-necked round-bottom flask, were placed tert-butyl (2R, 3S, 4R, 5S)-4-(2-bromo-4-chlorophenyl)-3-(3-chloro-2-fluorophenyl)-4-cyano-5-neopentylpyrrolidine-2-carboxylate (racemate) (4.8 g, 8.2 mmol, 1.0 eq) and BH₃ (10 M in dimethyl sulfide, 50 mL). The reaction mixture was stirred for 1 hour at 50° C. The resulting mixture was diluted with tetrahydrofuran (500 mL), and then quenched by the addition of CH₃OH (100 mL). The resulting solution was concentrated under vacuum. The crude residue was applied onto a silica gel column and eluted with ethyl acetate/petroleum ether=(1:0 to 10:1) to give tert-butyl (2R, 3S, 4R, 5S)-4-(aminomethyl)-4-(2-bromo-4-chlorophenyl)-3-(3-chloro-2-fluorophenyl)-5-neopentylpyrrolidine-2-carboxylate (racemate) (350 mg, 7%) as an off white solid. LC-MS (ESI, m/z) M+1: 587/589. ¹HNMR (300 MHz, DMSO-d₆) δ 8.14 (br, 3H), 7.82 (s, 1H), 7.56 (br, 3H), 7.32-7.15 (m, 2H), 4.85 (br, 1H), 4.42-4.39 (m, 2H), 3.70 (br, 1H), 3.49 (br, 1H), 1.61-1.58 (m, 1H), 1.38 (d, J=14.1 Hz, 1H), 1.24 (s, 9H), 0.99 (s, 9H).

### Synthesis of tert-butyl (2'S,3S,4'S,5′R)-6-chloro-4′-(3-chloro-2-fluorophenyl)-2′-neopentylspiro [indoline-3,3′-pyrrolidine]-5′-carboxylate (racemate)

Into a 40 mL vial purged and maintained under an inert atmosphere of nitrogen, were placed tert-butyl (2R, 3S, 4R, 5S and 2S, 3R, 4S, 5R)-4-(aminomethyl)-4-(2-bromo-4-chlorophenyl)-3-(3-chloro-2-fluorophenyl)-5-neopentylpyrrolidine-2-carboxylate (racemate) (300 mg, 0.5 mmol, 1.0 eq), toluene (12 mL), CuI (19 mg, 0.1 mmol, 0.2 eq), trans-1,2-diaminocyclohexane (12 mg, 0.1 mmol, 0.2 eq), K₃PO₄ (217 mg, 1.0 mmol, 2.0 eq). The reaction mixture was stirred for 16 hours at 100° C. Then the resulting solution was diluted with water (30 mL) and extracted with ethyl acetate (2×50 mL). The combined organic phase was washed with brine (2×50 mL), dried over anhydrous sodium sulfate. After filtration, the filtrate was concentrated under vacuum. The crude residue was applied onto a silica gel column and eluted with ethyl acetate/petroleum ether=(0:1 to 1:1) to give tert-butyl (2'S,3S,4'S,5′R)-6-chloro-4′-(3-chloro-2-fluorophenyl)-2′-neopentylspiro[indoline-3,3′-pyrrolidine]-5′-carboxylate (racemate) (130 mg, 50%) as a light yellow oil. LC-MS (ESI, m/z) M+1: 507/509. ¹HNMR (300 MHz, Methanol-d₄) δ 7.36-7.31 (m, 1H), 7.28 (d, J=6.6 Hz, 1H), 7.14-7.12 (m, 2H), 6.65 (d, J=7.8 Hz, 1H), 6.44 (s, 1H), 4.12 (d, J=9.6 Hz, 1H), 3.99 (d, J=9.6 Hz, 1H), 3.60 (d, J=10.5 Hz, 1H), 3.50 (d, J=7.5 Hz, 1H), 3.47 (d, J=10.5 Hz, 1H), 1.41 (d, J=10.2 Hz, 2H), 1.33 (s, 9H), 0.88 (s, 9H).

### Synthesis of (2'S,3S,4'S,5′R)-6-chloro-4′-(3-chloro-2-fluorophenyl)-2′-neopentylspiro[indoline-3,3′-pyrrolidine]-5′-carboxylic acid (racemate)

Into a 250 mL round-bottom flask were placed tert-butyl (2'S,3S,4'S,5′R)-6-chloro-4′-(3-chloro-2-fluorophenyl)-2′-neopentylspiro[indoline-3,3′-pyrrolidine]-5′-carboxylate (racemate) (5.5 g, 10.8 mmol, 1.0 eq), trifluoroacetic acid (60 mL). The reaction mixture was stirred for 16 hours at 25° C. The resulting mixture was diluted with water (200 mL) and then extracted with ethyl acetate (2×100 mL). The combined organic phase was washed with brine (2×100 mL), dried over anhydrous sodium sulfate. After filtration, the filtrate was concentrated under vacuum. Finally, (2'S,3S,4'S,5′R)-6-chloro-4′-(3-chloro-2-fluorophenyl)-2′-neopentylspiro[indoline-3,3′-pyrrolidine]-5′-carboxylic acid (racemate) (4.7 g, crude) was obtained as light yellow solid. LC-MS (ESI, m/z) M+1: 451/453.

### Example INT-4: Preparation of tert-butyl (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylate (racemate)

### Synthesis of 4-chloro-6-(trifluoromethyl)pyridine-3-carboxylic acid

To a solution of 2,2,6,6-tetramethylpiperidine (33.7 g, 235.5 mmol, 3.0 eq) in tetrahydrofuran (300 mL) was added n-BuLi (2.5M in hexanes, 95 mL) dropwise at −78° C. Then the reaction mixture was stirred at −78° C.˜−30° C. for 30 minutes. After that, the mixture was cooled to −78° C. and a solution of 6-(trifluoromethyl)pyridine-3-carboxylic acid (15.0 g, 78.5 mmol, 1.0 eq) in tetrahydrofuran (300 mL) was added dropwise at −78° C., and the mixture was stirred at −78° C.˜−40° C. for another 1 hour. The mixture was cooled to −78° C. and a solution of hexachloroethane (38.1 g, 160.9 mmol, 2.1 eq) in tetrahydrofuran (100 mL) was added to the above solution dropwised. The reaction mixture was stirred at −78° C. for 3 hours. The resulting mixture was quenched by the addition of NH₄Cl (aq.) (300 mL) at −78° C., acidified to pH 3-4 with HCl (1 M) and then extracted with ethyl acetate (2×300 mL). The combined organic phase was washed with brine (2×300 mL), dried over anhydrous sodium sulfate. After filtration, the filtrate was concentrated under vacuum. The crude residue was applied onto a silica gel column and eluted with ethyl acetate/petroleum ether=(1:0 to 20:1) to give 4-chloro-6-(trifluoromethyl)pyridine-3-carboxylic acid (10.0 g, 56%) as a light yellow solid. LC-MS (ESI, m/z) M-1: 224/226. ¹HNMR (400 MHz, Methanol-d₄) δ 9.10 (d, J=17.2 Hz, 1H), 8.04 (s, 1H).

### Synthesis of [4-chloro-6-(trifluoromethyl)pyridin-3-yl]methanol

Into a 500-mL 3-necked round-bottom flask, were placed 4-chloro-6-(trifluoromethyl)pyridine-3-carboxylic acid (10.0 g, 44.3 mmol, 1.0 eq), tetrahydrofuran (100 mL). After that, BH₃-tetrahydrofuran (1 M in tetrahydrofuran, 178 mL) was added at 0° C. The reaction mixture was stirred for 16 hours at 25° C. The resulting mixture was quenched by the addition of NH₄Cl (aq.) (250 mL), and then extracted with ethyl acetate (2×200 mL). The combined organic phase was washed with brine (2×200 mL), dried over anhydrous sodium sulfate. After filtration, the filtrate was concentrated under vacuum to give [4-chloro-6-(trifluoromethyl)pyridin-3-yl]methanol (12.0 g, crude) as a light yellow oil. LC-MS (ESI, m/z) M+1: 212/214. ¹HNMR (400 MHz, DMSO-d₆) δ 8.82 (s, 1H), 8.07 (s, 1H), 5.73 (t, J=5.6 Hz, 1H). 4.70 (d, J=5.6 Hz, 2H).

### Synthesis of 4-chloro-5-(chloromethyl)-2-(trifluoromethyl)pyridine

[4-chloro-6-(trifluoromethyl)pyridin-3-yl]methanol (12.0 g, 56.7 mmol, 1.0 eq) was added to SOCl₂ (120 mL) slowly with ice bath, the reaction mixture was stirred for 16 hours at 85° C. The resulting mixture was concentrated and the residue was poured into water (200 mL). The resulting aqueous solution was adjusted to pH=7-8 with solid NaHCO₃, and then extracted with ethyl acetate (2×300 mL). The combined organic phase was washed with brine (2×300 mL), dried over anhydrous sodium sulfate. After filtration, the filtrate was concentrated under vacuum to give 4-chloro-5-(chloromethyl)-2-(trifluoromethyl)pyridine (13.0 g, crude) as a yellow oil.

### Synthesis of 2-[4-chloro-6-(trifluoromethyl)pyridin-3-yl]acetonitrile

Into a 500-mL round-bottom flask were placed 4-chloro-5-(chloromethyl)-2-(trifluoromethyl)pyridine (13.0 g, 56.5 mmol, 1.0 eq), TBAF (29.6 g, 113.0 mmol, 2.0 eq), CH₃CN (150 mL), TMSCN (11.2 g, 113.0 mmol, 2.0 eq). The reaction mixture was stirred for 2 hours at 25° C. The resulting mixture was concentrated under vacuum. The residue was diluted with water (200 mL) and then extracted with ethyl acetate (2×200 mL). The combined organic phases was washed with brine (2×200 mL), dried over anhydrous sodium sulfate. After filtration, the filtrate was concentrated in vacuo. The crude residue was applied onto a silica gel column and eluted with ethyl acetate/petroleum ether=(0:1 to 1:3) to give 2-[4-chloro-6-(trifluoromethyl)pyridin-3-yl]acetonitrile (1.5 g, 12%) as a light yellow oil. LC-MS (ESI, m/z) M+1: 221/223. ¹HNMR (300 MHz, Chloroform-d) δ 8.85 (s, 1H), 7.81 (s, 1H), 3.94 (s, 2H).

### Synthesis of 3-(3-chloro-2-fluorophenyl)-2-(4-chloro-6-(trifluoromethyl)pyridin-3-yl)acrylonitrile

Into a 40 mL sealed tube, were placed 2-[4-chloro-6-(trifluoromethyl)pyridin-3-yl]acetonitrile (1.2 g, 5.4 mmol, 1.0 eq), CH₃OH (12 mL), 3-chloro-2-fluorobenzaldehyde (0.9 g, 5.4 mmol, 1.0 eq) and piperidine (0.7 g, 8.2 mmol, 1.5 eq). The reaction mixture was stirred for 3 hours at 50° C. The resulting mixture was concentrated under vacuum. The residue was applied onto a silica gel column and eluted with ethyl acetate/petroleum ether=(0:1 to 1:5) to give 3-(3-chloro-2-fluorophenyl)-2-(4-chloro-6-(trifluoromethyl)pyridin-3-yl)acrylonitrile (800 mg, 41%) as a yellow solid. ¹HNMR (300 MHz, DMSO-d₆) δ 9.0 (s, 1H), 8.36 (s, 1H), 8.10-8.03 (m, 1H), 7.99 (s, 1H), 7.88-7.81 (m, 1H), 7.49 (td, J=7.8, 2.1 Hz, 1H).

### Synthesis of tert-butyl (2R,3S,4R,5S)-3-(3-chloro-2-fluorophenyl)-4-[4-chloro-6-(trifluoromethyl) pyridin-3-yl]-4-cyano-5-(2,2-dimethylpropyl)pyrrolidine-2-carboxylate (racemate)

Into a 50-mL round-bottom flask, were placed 3-(3-chloro-2-fluorophenyl)-2-(4-chloro-6-(trifluoromethyl)pyridin-3-yl)acrylonitrile (800 mg, 2.2 mmol, 1.0 eq), tert-butyl 2-((3,3-dimethylbutylidene)amino)acetate (709 mg, 3.3 mmol, 1.5 eq), AgF (337 mg, 2.6 mmol, 1.2 eq), triethylamine (448 mg, 4.4 mmol, 2.0 eq) and 1,2-dichloroethane (10 mL). The reaction mixture was stirred for 16 hours at 25° C. The resulting mixture was filtered and the filtrate was concentrated under vacuum. The residue was applied onto a silica gel column and eluted with ethyl acetate/petroleum ether=(0:1 to 1:3) to give tert-butyl (2R,3S,4R,5S)-3-(3-chloro-2-fluorophenyl)-4-[4-chloro-6-(trifluoromethyl)pyridin-3-yl]-4-cyano-5-(2,2-dimethylpropyl)pyrrolidine-2-carboxylate (racemate) (400 mg, 31%) as an off white solid. LC-MS (ESI, m/z) M+1: 574/576. ¹HNMR (400 MHz, DMSO-d₆) δ 8.74 (s, 1H), 8.34 (s, 1H), 7.75 (t, J=6.8 Hz, 1H), 7.56 (t, J=7.2 Hz, 1H), 7.36 (t, J=8.0 Hz, 1H), 5.03 (d, J=7.0 Hz, 1H), 4.56 (t, J=9.4 Hz, 1H), 4.37 (t, J=7.0 Hz, 1H), 3.63-3.54 (m, 1H), 1.54 (dd, J=14.2, 9.6 Hz, 1H), 1.34 (s, 9H), 1.29 (d, J=7.8 Hz, 1H), 0.91 (s, 9H).

### Synthesis of tert-butyl (2R,3S,4S,5S)-4-(aminomethyl)-3-(3-chloro-2-fluorophenyl)-4-[4-chloro-6-(trifluoromethyl)pyridin-3-yl]-5-(2,2-dimethylpropyl)pyrrolidine-2-carboxylate (racemate)

Into a 50-mLround-bottom flask, were placed tert-butyl (2R,3S,4R,5S)-3-(3-chloro-2-fluorophenyl)-4-[4-chloro-6-(trifluoromethyl)pyridin-3-yl]-4-cyano-5-(2,2-dimethylpropyl)pyrrolidine-2-carboxylate (racemate) (400 mg, 0.7 mmol, 1.0 eq), CH₃OH (9 mL), acetic acid (3 mL) and Raney-Ni (40 mg, 0.5 mmol, 0.7 eq). The reaction mixture was stirred at 25° C. under hydrogen (2 atm) for 24 hours. The resulting mixture was filtered and the filtrate was concentrated under vacuum. Finally, tert-butyl (2R,3S,4S,5S)-4-(aminomethyl)-3-(3-chloro-2-fluorophenyl)-4-[4-chloro-6-(trifluoromethyl)pyridin-3-yl]-5-(2,2-dimethylpropyl)pyrrolidine-2-carboxylate (racemate) (600 mg, crude) was obtained as a light green oil. LC-MS (ESI, m/z) M+1: 578/579.

### Synthesis of tert-butyl (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylate (racemate)

Into a 50 mL round-bottom flask were placed tert-butyl (2R,3S,4S,5S)-4-(aminomethyl)-3-(3-chloro-2-fluorophenyl)-4-[4-chloro-6-(trifluoromethyl)pyridin-3-yl]-5-(2,2-dimethylpropyl)pyrrolidine-2-carboxylate (racemate) (600 mg, 1.0 mmol, 1.0 eq), CH₃CN (10 mL), K₂CO₃ (717 mg, 5.1 mmol, 5.0 eq).

The reaction mixture was stirred for 16 hours at 25° C. The resulting mixture was filtered, the filtrate was concentrated under vacuum. The residue was diluted with water (50 mL) and extracted with ethyl acetate (50 mL). The organic phase was washed with brine (2×50 mL), dried over anhydrous sodium sulfate. After filtration, the filtrate was concentrated under vacuum. The crude residue was applied onto a silica gel column and eluted with ethyl acetate/petroleum ether=(0:1 to 1:0) to give tert-butyl (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylate (racemate) (180 mg) as a light yellow solid. LC-MS (ESI, m/z) M+1: 542/543. ¹HNMR (300 MHz, DMSO-d₆) δ 8.27 (s, 1H), 7.51-7.39 (m, 2H), 7.27-7.15 (m, 1H), 6.63 (d, J=15.0 Hz, 2H), 4.12 (s, 1H), 3.62 (d, J=12.0 Hz, 1H), 3.48-3.34 (m, 2H), 2.95 (s, 1H), 1.27 (s, 9H), 1.14 (d, J=13.8 Hz, 2H), 0.82 (s, 9H).

### Example INT-5: Preparation of tert-butyl (2S,3S,4S,5R)-6′-chloro-4-(3-chloro-2-fluorophenyl)-2-neopentyl-1′,2′-dihydrospiro[pyrrolidine-3,3′-pyrrolo[3,2-c]pyridine]-5-carboxylate

### Synthesis of 5-(bromomethyl)-2,4-dichloropyridine

Into a 500 mL 3-necked round-bottom flask were added 2,4-dichloro-5-methylpyridine (25.0 g, 154.3 mmol, 1.0 eq), chlorobenzene (250 mL), AlBN (2.5 g, 15.4 mmol, 0.1 eq) and NBS (30.1 g, 169.7 mmol, 1.1 eq) at 25° C. The reaction mixture was stirred for 16 hours at 130° C. The resulting mixture was concentrated under vacuum. The residue was diluted with water (100 mL), and extracted with ethyl acetate (3×100 mL). The combined organic layers were washed with brine (100 mL), dried over anhydrous sodium sulfate. After filtration, the filtrate was concentrated under vacuum. The crude residue was applied onto a silica gel column and eluted with ethyl acetate/petroleum ether=10:1 to give 5-(bromomethyl)-2,4-dichloropyridine (16.3 g, 43%) as white solid. LC-MS (ESI, m/z) M+1: 240/242.

### Synthesis of 2-(4,6-dichloropyridin-3-yl)acetonitrile

Into a 500 mL 3-necked round-bottom flask were added 5-(bromomethyl)-2,4-dichloropyridine (16.3 g, 67.6 mmol, 1.0 eq), acetonitrile (160 mL) and LiOH·H₂O (1.9 g, 81.2 mmol, 1.2 eq) at 25° C. To the above mixture was added TMSCN (8.1 g, 81.2 mmol, 1.2 eq) dropwise at 25° C. The reaction mixture was stirred for additional 2 hours at 25° C. The resulting mixture was diluted with water (200 mL) and extracted with ethyl acetate (3×100 mL). The combined organic layers were washed with brine (2×100 mL), dried over anhydrous sodium sulfate. After filtration, the filtrate was concentrated under vacuum. The crude residue was applied onto a silica gel column and eluted with ethyl acetate/petroleum ether=(10:1 to 3:1) to give 2-(4,6-dichloropyridin-3-yl)acetonitrile (12.0 g, 94%) as a white solid. LC-MS (ESI, m/z) M+1: 187/189. ¹HNMR (300 MHz, DMSO-d₆) δ 8.53 (s, 1H), 7.93 (s, 1H), 4.16 (s, 2H).

### Synthesis of 3-(3-chloro-2-fluorophenyl)-2-(4,6-dichloropyridin-3-yl)prop-2-enenitrile

Into a 500 mL 3-necked round-bottom flask were added 2-(4,6-dichloropyridin-3-yl)acetonitrile (12.0 g, 64.2 mmol, 1.0 eq), 1,2-dichloroethane (200 mL), 3-chloro-2-fluorobenzaldehyde (11.2 g, 70.6 mmol, 1.1 eq) and Cs₂CO₃ (41.8 g, 128.3 mmol, 2.0 eq) at 25° C. The reaction mixture was stirred for 3 hours at 70° C. The resulting mixture was diluted with water (200 mL) and then extracted with dichloromethane (2×200 mL). The combined organic layers were washed with brine (300 mL), dried over anhydrous sodium sulfate. After filtration, the filtrate was concentrated under vacuum. The crude residue was applied onto a silica gel column and eluted with ethyl acetate/petroleum ether=(10:1 to 3:1) to give 3-(3-chloro-2-fluorophenyl)-2-(4,6-dichloropyridin-3-yl)prop-2-enenitrile (10.0 g, 47%) as a white solid. LC-MS (ESI, m/z) M+1: 327/329. ¹HNMR (400 MHz, DMSO-d₆) δ 8.67 (s, 1H), 8.08-7.99 (m, 2H), 7.89 (s, 1H), 7.82 (t, J=7.5 Hz, 1H), 7.47 (t, J=8.0 Hz, 1H).

### Synthesis of tert-butyl (2S,3S,4S,5R)-3-(3-chloro-2-fluorophenyl)-4-cyano-4-(4,6-dichloropyridin-3-yl)-5-(2,2-dimethylpropyl)pyrrolidine-2-carboxylate (racemate)

Into a 500 mL round-bottom flask were added 3-(3-chloro-2-fluorophenyl)-2-(4,6-dichloropyridin-3-yl)prop-2-enenitrile (10.0 g, 30.5 mmol, 1.0 eq), 1,2-dichloroethane (200 mL), tert-butyl 2-[(3,3-dimethylbutylidene)amino]acetate (11.7 g, 55.0 mmol, 1.8 eq), trimethylamine (6.2 g, 61.1 mmol, 2.0 eq) and AgF (4.6 g, 36.6 mmol, 1.2 eq) at 25° C. The reaction mixture was stirred for 16 hours at 25° C. under nitrogen atmosphere. The resulting mixture was diluted with brine (200 mL) and then extracted with dichloromethane (2×200 mL). The combined organic layers were washed with brine (200 mL), dried over anhydrous sodium sulfate. After filtration, the filtrate was concentrated under vacuum. The crude residue was applied onto a silica gel column and eluted with ethyl acetate/petroleum ether=10:1 to give tert-butyl (2S,3S,4S,5R)-3-(3-chloro-2-fluorophenyl)-4-cyano-4-(4,6-dichloropyridin-3-yl)-5-(2,2-dimethylpropyl)pyrrolidine-2-carboxylate (racemate) (2.8 g, 17%) as a light yellow solid. LC-MS (ESI, m/z) M+1: 540/542. ¹HNMR: (300 MHz, DMSO-d₆) δ 8.38 (s, 1H), 8.03 (s, 1H), 7.72 (t, J=7.2 Hz, 1H), 7.56 (td, J=7.5, 1.2 Hz, 1H), 7.35 (t, J=8.0 Hz, 1H), 4.99 (d, J=7.1 Hz, H), 4.47 (t, J=9.2 Hz, 1H), 4.34 (t, J=6.8 Hz, 1H), 3.58-3.46 (m, 1H), 1.51-1.47 (m, 1H), 1.34 (s, 9H), 1.24 (d, J=4.3 Hz, 1H), 0.90 (s, 9H).

### Synthesis of tert-butyl (2S,3S,4S,5R)-4-(aminomethyl)-3-(3-chloro-2-fluorophenyl)-4-(4,6-dichloropyridin-3-yl)-5-(2,2-dimethylpropyl)pyrrolidine-2-carboxylate (racemate)

Into a 250 mL 3-necked round-bottom flask were added methanol (30 mL), acetic acid (10 mL), Raney Ni (0.9 g, 15.5 mmol, 3.0 eq) and tert-butyl (2S,3S,4S,5R)-3-(3-chloro-2-fluorophenyl)-4-cyano-4-(4,6-dichloropyridin-3-yl)-5-(2,2-dimethylpropyl)pyrrolidine-2-carboxylate (racemate) (2.8 g, 5.2 mmol, 1.0 eq) at 25° C. The reaction mixture was stirred for 6 hours at 25° C. under hydrogen (3 atm) atmosphere. The resulting mixture was filtered and the filter cake was washed with methanol (60 mL). The filtrate was concentrated under vacuum to give tert-butyl (2S,3S,4S,5R)-4-(aminomethyl)-3-(3-chloro-2-fluorophenyl)-4-(4,6-dichloropyridin-3-yl)-5-(2,2-dimethylpropyl)pyrrolidine-2-carboxylate (racemate) (2.4 g, crude) as colorless oil. LC-MS (ESI, m/z) M+1: 544/546.

### Synthesis of tert-butyl (2S,3S,4S,5R)-6′-chloro-4-(3-chloro-2-fluorophenyl)-2-neopentyl-1′,2′-dihydrospiro[pyrrolidine-3,3′-pyrrolo[3,2-c]pyridine]-5-carboxylate (racemate)

Into a 100 mL round-bottom flask were added tert-butyl (2S,3S,4S,5R)-4-(aminomethyl)-3-(3-chloro-2-fluorophenyl)-4-(4,6-dichloropyridin-3-yl)-5-(2,2-dimethylpropyl)pyrrolidine-2-carboxylate (racemate) (2.4 g, 4.4 mmol, 1.0 eq), acetonitrile (25 mL) and K₂CO₃ (1.2 g, 8.8 mmol, 2.0 eq) at 25° C. The reaction mixture was stirred for 3 hours at 70° C. The resulting mixture was diluted with water (30 mL) and extracted with ethyl acetate (3×30 mL). The combined organic layers were washed with brine (30 mL), dried over anhydrous sodium sulfate. After filtration, the filtrate was concentrated under vacuum. The crude residue was applied onto a silica gel column and eluted with ethyl acetate/petroleum ether=(10:1 to 1:1) to give tert-butyl (2S,3S,4S,5R)-6′-chloro-4-(3-chloro-2-fluorophenyl)-2-neopentyl-1′,2′-dihydrospiro[pyrrolidine-3,3′-pyrrolo[3,2-c]pyridine]-5-carboxylate (racemate) (170 mg, 7%) as light yellow solid. LC-MS (ESI, m/z) M+1: 508/510. ¹HNMR (300 MHz, DMSO-d₆) δ 7.92 (d, J=1.6 Hz, 1H), 7.44 (t, J=7.4 Hz, 2H), 7.20 (t, J=7.9 Hz, 1H), 6.88 (s, 1H), 6.56 (s, 1H), 6.21 (s, 1H), 4.07 (s, 2H), 3.57 (d, J=11.1 Hz, 1H), 3.36-3.34 (m, 1H), 2.90 (bs, 1H), 2.18 (s, 1H), 1.43-1.14 (m, 2H), 1.25 (s, 9H), 0.83 (s, 9H).

### Example INT-6: Preparation of 3-(4-iodo-1-oxo-3H-isoindol-2-yl)piperidine-2,6-dione

### Synthesis of methyl 3-iodo-2-methylbenzoate

To a stirred mixture of 3-iodo-2-methylbenzoic acid (20.0 g, 76.3 mmol, 1.0 eq) in methanol (200 mL) was added thionyl chloride (27.2 g, 228.9 mmol, 3.0 eq) dropwise at 0° C. The reaction mixture was stirred for 3 hours at 80° C. The resulting mixture was neutralized to pH=7 with saturated NaHCO₃ (aq.) and then extracted with ethyl acetate (3×200 mL). The combined organic phase was washed with brine (200 mL), dried over anhydrous sodium sulfate. After filtration, the filtrate was concentrated under vacuum to give methyl 3-iodo-2-methylbenzoate (18.0 g, 85%) as yellow oil. ¹HNMR (300 MHz, DMSO-d₆) δ 8.06 (dd, J=7.8, 1.2 Hz, 1H), 7.70 (dd, J=7.8, 1.5 Hz, 1H), 7.06 (td, J=7.8, 0.6 Hz, 1H), 3.84 (s, 3H), 2.55 (s, 3H).

### Synthesis of 3-(4-iodo-1-oxo-3H-isoindol-2-yl)piperidine-2,6-dione

A mixture of methyl 3-iodo-2-methylbenzoate (18.0 g, 65.2 mmol, 1.0 eq), NBS (13.9 g, 78.2 mmol, 1.2 eq) and AlBN (1.1 g, 6.5 mmol, 0.1 eq) in CCl₄ (200 mL) was stirred for 14 hours at 80° C. The resulting mixture was concentrated under vacuum, and then diluted with dichloromethane (200 mL). The organic phase was washed with water (3×50 mL), brine (50 mL) and dried over anhydrous sodium sulfate. After filtration, the filtrate was concentrated under vacuum to give methyl 2-(bromomethyl)-3-iodobenzoate (16.0 g, 69%) as a brown solid.

### Synthesis of 3-(4-iodo-1-oxo-3H-isoindol-2-yl)piperidine-2,6-dione

A mixture of methyl 2-(bromomethyl)-3-iodobenzoate (16.0 g, 45.1 mmol, 1.0 eq), 3-aminopiperidine-2,6-dione (8.7 g, 67.6 mmol, 1.5 eq) and triethylamine (13.7 g, 135.2 mmol, 3.0 eq) in acetonitrile (150 mL) was stirred for 14 hours at 80° C. The resulting mixture was concentrated under vacuum, diluted with ethyl acetate (100 mL) and water (100 mL). The precipitated solids were collected by filtration and washed with water (50 mL). Finally, 3-(4-iodo-1-oxo-3H-isoindol-2-yl)piperidine-2,6-dione (11.0 g, 66%) was obtained as a blue solid. ¹HNMR (300 MHz, DMSO-d₆) δ 11.02 (bs, 1H), 7.88 (d, J=7.8 Hz, 1H), 7.78 (d, J=7.5 Hz, 1H), 7.52 (t, J=7.8 Hz, 1H), 5.16 (dd, J=13.2, 5.1 Hz, 1H), 4.43 (d, J=17.7 Hz, 1H), 4.27 (d, J=17.7 Hz, 1H), 2.93 (ddd, J=18.3, 13.5, 5.4 Hz, 1H), 2.66-2.52 (m, 1H), 2.44 (dd, J=13.5, 4.5 Hz, 1H), 2.11-1.96 (m, 1H).

### Example INT-12: Preparation of 3-(5-iodo-1-oxoisoindolin-2-yl)piperidine-2,6-dione

### Synthesis of methyl 2-(bromomethyl)-4-iodobenzoate

Into a 500-mL round-bottom flask, were placed methyl 4-iodo-2-methylbenzoate (24.0 g, 86.9 mmol, 1.0 eq), NBS (20.1 g, 113.0 mmol, 1.3 eq), AlBN (8.6 g, 52.2 mmol, 0.6 eq), CCl₄ (250 mL). The reaction mixture was stirred for 14 hours at 80° C. The resulting mixture was then quenched by the addition of water (100 mL) and then extracted with dichloromethane (2×100 mL). The combined organics were washed with brine (2×100 mL) and dried over anhydrous Na₂SO₄. After filtration, the filtrate was concentrated under vacuum to give methyl 2-(bromomethyl)-4-iodobenzoate as a dark blue solid (26.0 g crude).

### Synthesis of 3-(5-iodo-1-oxoisoindolin-2-yl)piperidine-2,6-dione

Into a 1000-mL round-bottom flask, were placed methyl 2-(bromomethyl)-4-iodobenzoate (26.0 g, 73.2 mmol, 1.0 eq), 3-aminopiperidine-2,6-dione (14.1 g, 109.9 mmol, 1.5 eq), Et₃N (22.2 g, 219.7 mmol, 3.0 eq), CH₃CN (300 mL). The resulting solution was stirred for 14 hours at 80° C. The resulting mixture was then quenched by the addition of water (100 mL) and then extracted with ethyl acetate (2×100 mL). The combined organics were washed with brine (2×100 mL) and dried over anhydrous Na₂SO₄. After filtration, the resulting mixture was concentrated under vacuum. The crude residue was applied onto a silica gel column and eluted with ethyl acetate/petroleum ether=1:0 to give 3-(5-iodo-1-oxoisoindolin-2-yl)piperidine-2,6-dione as a dark blue solid (5.4 g, 19.9%). LC-MS (ESI, m/z) M+1: 370. ¹HNMR (300 MHz, DMSO-d₆) δ 11.00 (s, 1H), 8.07 (s, 1H), 7.89 (d, J=7.8 Hz, 1H), 7.52 (d, J=9.0 Hz, 1H), 5.11 (dd, J=13.5, 5.1 Hz, 1H), 4.45 (d, J=17.7 Hz, 1H), 4.32 (d, J=17.4 Hz, 1H), 2.99-2.79 (m, 1H), 2.60 (d, J=17.7 Hz, 1H), 2.45-2.28 (m, 1H), 2.02 (d, J=8.7 Hz, 1H).

### Example INT-15: Preparation of (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylic acid

### Synthesis of (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylic acid

Into a 50 mL round-bottom flask, were placed tert-butyl (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylate (300 mg, 0.6 mmol, 1.0 eq), trifluoroacetic acid (6 mL). The resulting solution was stirred for 16 hours at 25° C. The reaction was quenched by the addition of water (50 mL) and then extracted with ethyl acetate (2×50 mL). The combined organics were washed with water (2×50 mL) and brine (2×50 mL). The mixture was dried over anhydrous Na₂SO₄. After filtration, the filtrate was concentrated under vacuum to give (2S,3S,4S,5R)-4-(3-chloro-2-fluorophenyl)-2-(2,2-dimethylpropyl)-6′-(trifluoromethyl)-1′,2′-dihydrospiro [pyrrolidine-3,3′-pyrrolo[3,2-c]pyridine]-5-carboxylic acid as an off-white solid (200 mg, crude). ¹HNMR (400 MHz, Methanol-d4,) 5 8.60 (s, 1H), 7.59 (t, J=7.0 Hz, 1H), 7.47 (t, J=7.6 Hz, 1H), 7.25 (t, J=8.0 Hz, 1H), 6.88 (s, 1H), 5.14 (d, J=11.4 Hz, 1H), 4.55 (d, J=11.4 Hz, 1H), 4.37 (d, J=8.0 Hz, 1H), 4.03 (d, J=12.6 Hz, 1H), 3.87 (d, J=12.6 Hz, 1H), 1.96 (dd, J-15.4, 8.6 Hz, 1H), 1.65 (d, J=15.4 Hz, 1H), 0.99 (s, 9H).

### Example INT-17: Preparation of 2-(2,6-dioxopiperidin-3-yl)-5-(piperazin-1-yl)isoindoline-1,3-dione

### Synthesis of tert-butyl 4-(2-(2,6-dioxopiperidin-3-yl)-1,3-dioxoisoindolin-5-yl)piperazine-1-carboxylate

Into a 100 mL round-bottom flask were added 2-(2,6-dioxopiperidin-3-yl)-5-fluoroisoindoline-1,3-dione (2.0 g, 7.2 mmol, 1.0 eq), tert-butyl piperazine-1-carboxylate (1.3 g, 7.2 mmol, 1.0 eq), TEA (1.4 g, 14.4 mmol, 2.0 eq) and DMSO (20 mL). The resulting mixture was stirred for 2 hours at 120° C. under nitrogen atmosphere. The mixture was diluted with water (60 mL) and then extracted with CH₂Cl₂ (2×50 mL). The combined organic layers were washed with brine (50 mL), dried over anhydrous Na₂SO₄. After filtration, the filtrate was concentrated under reduced pressure to afford tert-butyl 4-(2-(2,6-dioxopiperidin-3-yl)-1,3-dioxoisoindolin-5-yl)piperazine-1-carboxylate (2.8 g, 87.0%) as a brown solid. ¹HNMR (500 MHz, DMSO-d₆) δ 11.05 (s, 1H), 7.70-7.68 (d, J=10.0 Hz, 1H), 7.34 (s, 1H), 7.25-7.23 (d, J=10.0 Hz, 1H), 5.09-5.05 (m, 1H), 2.88 (m, 1H), 2.61-2.52 (m, 2H), 2.02 (m, 1H), 1.43 (s, 9H).

### Synthesis of 2-(2,6-dioxopiperidin-3-yl)-5-(piperazin-1-yl)isoindoline-1,3-dione

Into a 100 mL round-bottom flask were added tert-butyl 4-(2-(2,6-dioxopiperidin-3-yl)-1,3-dioxoisoindolin-5-yl)piperazine-1-carboxylate (2.8 g, 8.1 mmol, 1.0 eq) and HCl/dioxane (28 mL, 4 M). The mixture was stirred for 3 hours at 25° C. The resulting mixture was concentrated under reduced pressure to afford 2-(2,6-dioxopiperidin-3-yl)-5-(piperazin-1-yl)isoindoline-1,3-dione (2.3 g, 96%) as a white solid.

### Example 71: Preparation of ((2S,3S,4S,5R)-4-(3-chloro-2-fluorophenyl)-2-(2,2-dimethylpropyl)-N-(4-{4-[2-(2,6-dioxopiperidin-3-yl)-1-oxo-3H-isoindol-5-yl]piperazine-1-carbonyl}-2-methoxyphenyl)-6′-(trifluoromethyl)-1′,2′-dihydrospiro[pyrrolidine-3,3′-pyrrolo[3,2-c]pyridine]-5-carboxamide

### Synthesis of tert-butyl 4-[2-(2,6-dioxopiperidin-3-yl)-1-oxo-3H-isoindol-5-yl]piperazine-1-carboxylate

Into a 40-mL sealed-tube purged and maintained with an inert atmosphere of nitrogen, were placed 3-(5-iodo-1-oxo-3H-isoindol-2-yl)piperidine-2,6-dione (1.0 g, 2.7 mmol, 1.0 eq), tert-butyl piperazine-1-carboxylate (600 mg, 3.2 mmol, 1.2 eq), Cs₂CO₃ (1.8 g, 5.4 mmol, 2.0 eq), DMF (10 mL), 3rd Generation RuPhos precatalyst (230 mg, 0.3 mmol, 0.1 eq). The resulting solution was stirred for 14 hours at 110° C. The resulting mixture was then quenched by the addition of water (40 mL) and then extracted with ethyl acetate (2×40 mL). The combined organics were washed with brine (2×40 mL) and dried over anhydrous Na₂SO₄. After filtration, the filtrate was concentrated under vacuum. The crude residue was applied onto a silica gel column and eluted with ethyl acetate/petroleum ether=1:1 to give tert-butyl 4-[2-(2,6-dioxopiperidin-3-yl)-1-oxo-3H-isoindol-5-yl]piperazine-1-carboxylate as a light yellow solid (400 mg, 34.6%). LC-MS (ESI, m/z) M+1: 429. ¹HNMR (400 MHz, DMSO-d₆) δ 10.95 (s, 1H), 7.54 (d, J=8.4 Hz, 1H), 7.07 (d, J=11.2 Hz, 2H), 5.06 (dd, J=13.4, 5.2 Hz, 1H), 4.34 (d, J=16.8 Hz, 1H), 4.22 (d, J=16.8 Hz, 1H), 3.49-3.46 (m, 4H), 3.30-3.27 (m, 4H), 2.98-2.84 (m, 1H), 2.64-2.55 (m, 1H), 2.43-2.32 (m, 1H), 2.01-1.89 (m, 1H), 1.43 (s, 9H).

### Synthesis of 3-[1-oxo-5-(piperazin-1-yl)-3H-isoindol-2-yl]piperidine-2,6-dione hydrochloride

Into a 50-mL round-bottom flask, were placed tert-butyl 4-[2-(2,6-dioxopiperidin-3-yl)-1-oxo-3H-isoindol-5-yl]piperazine-1-carboxylate (380 mg, 0.9 mmol, 1.0 eq), CH₂Cl₂ (4 mL), HCl(gas) in 1,4-dioxane (4 mL). The resulting mixture was stirred for 1 hour at 25° C. The resulting mixture was concentrated under vacuum to give 3-[1-oxo-5-(piperazin-1-yl)-3H-isoindol-2-yl]piperidine-2,6-dione hydrochloride as a brown solid (350 mg crude). LC-MS (ESI, m/z) M+1: 329.

### Synthesis of 3-{5-[4-(3-methoxy-4-nitrobenzoyl)piperazin-1-yl]-1-oxo-3H-isoindol-2-yl}piperidine-2,6-dione

Into a 20-mL sealed tube, were placed 3-[1-oxo-5-(piperazin-1-yl)-3H-isoindol-2-yl]piperidine-2,6-dione hydrochloride (150 mg, 0.4 mmol, 1.0 eq), 3-methoxy-4-nitrobenzoic acid (89 mg, 0.5 mmol, 1.1 eq), HATU (172 mg, 0.5 mmol, 1.1 eq), DIEA (159 mg, 1.2 mmol, 3.0 eq), DMF (3 mL). The resulting solution was stirred for 2 hours at 25° C. The resulting mixture was then quenched by the addition of water (30 mL) and then extracted with ethyl acetate (2×30 mL). The combined organics were washed with brine (2×30 mL) and dried over anhydrous Na₂SO₄. After filtration, the filtrate was concentrated under vacuum. The crude residue was applied onto a silica gel column and eluted with ethyl acetate/petroleum ether=1:0 to give 3-{5-[4-(3-methoxy-4-nitrobenzoyl)piperazin-1-yl]-1-oxo-3H-isoindol-2-yl}piperidine-2,6-dione as a light yellow solid (160 mg, 76.7%). LC-MS (ESI, m/z) M+1: 508.

### Synthesis of 3-{5-[4-(4-amino-3-methoxybenzoyl)piperazin-1-yl]-1-oxo-3H-isoindol-2-yl}piperidine-2,6-dione

Into a 20-mL sealed tube, were placed 3-{5-[4-(3-methoxy-4-nitrobenzoyl)piperazin-1-yl]-1-oxo-3H-isoindol-2-yl}piperidine-2,6-dione (140 mg, 0.3 mmol, 1.0 eq), Fe (62 mg, 1.1 mmol, 4.0 eq), NH₄Cl (118 mg, 2.2 mmol, 8.0 eq), EtOH (3 mL), H₂O (1 mL). The resulting solution was stirred for 2 hours at 50° C. The resulting mixture was filtered, the filtrate was concentrated under vacuum. The crude residue was applied onto a silica gel column and eluted with ethyl acetate/petroleum ether=1:0 to give 3-{5-[4-(4-amino-3-methoxybenzoyl)piperazin-1-yl]-1-oxo-3H-isoindol-2-yl}piperidine-2,6-dione as a light yellow solid (110 mg, 83.5%). LC-MS (ESI, m/z) M+1: 478.

### Synthesis of ((2S,3S,4S,5R)-4-(3-chloro-2-fluorophenyl)-2-(2,2-dimethylpropyl)-N-(4-{4-[2-(2,6-dioxopiperidin-3-yl)-1-oxo-3H-isoindol-5-yl]piperazine-1-carbonyl}-2-methoxyphenyl)-6′-(trifluoromethyl)-1′,2′-dihydrospiro[pyrrolidine-3,3′-pyrrolo[3,2-c]pyridine]-5-carboxamide

Into an 8-mL sealed tube, were placed (2S,3S,4S,5R)-4-(3-chloro-2-fluorophenyl)-2-(2,2-dimethylpropyl)-6′-(trifluoromethyl)-1′,2′-dihydrospiro[pyrrolidine-3,3′-pyrrolo[3,2-c]pyridine]-5-carboxylic acid (30 mg, 0.1 mmol, 1.0 eq), 3-{5-[4-(4-amino-3-methoxybenzoyl)piperazin-1-yl]-1-oxo-3H-isoindol-2-yl}piperidine-2,6-dione (35 mg, 0.1 mmol, 1.2 eq), TCFH (26 mg, 0.1 mmol, 1.5 eq), NMI (13 mg, 0.1 mmol, 2.5 eq), CH₃CN (1 mL). The resulting solution was stirred for 2 hours at 25° C. The crude product was purified by Prep-HPLC using the following conditions: Column, SunFire Prep C18 OBD Column, 50*250 mm 5 μm 10 nm; mobile phase, Water (0.05% NH₃·H₂O) and CH₃CN (55% Phase B up to 65% in 7 min); Detector, UV 254/220 nm. Finally, ((2S,3S,4S,5R)-4-(3-chloro-2-fluorophenyl)-2-(2,2-dimethylpropyl)-N-(4-{4-[2-(2,6-dioxopiperidin-3-yl)-1-oxo-3H-isoindol-5-yl]piperazine-1-carbonyl}-2-methoxyphenyl)-6′-(trifluoromethyl)-1′,2′-dihydrospiro[pyrrolidine-3,3′-pyrrolo[3,2-c]pyridine]-5-carboxamide was obtained as a white solid (10 mg, 17.1%). LC-MS (ESI, m/z) M+1: 945/947. ¹HNMR (400 MHz, DMSO-d₆) δ 10.95 (s, 1H), 10.66 (s, 1H), 8.42 (d, J=1.6 Hz, 1H), 8.37 (d, J=8.2 Hz, 1H), 7.56 (d, J=8.4 Hz, 1H), 7.49-7.39 (m, 2H), 7.23 (t, J=8.0 Hz, 1H), 7.16 (d, J=1.8 Hz, 1H), 7.13-7.06 (m, 2H), 7.03 (dd, J=8.2, 1.8 Hz, 1H), 6.62 (d, J=2.4 Hz, 2H), 5.06 (dd, J=13.2, 5.2 Hz, 1H), 4.41-4.31 (m, 2H), 4.29-4.18 (m, 2H), 3.89 (s, 3H), 3.78 (t, J=11.2 Hz, 1H), 3.72-3.63 (m, 4H), 3.36 (d, J=11.4 Hz, 5H), 2.91 (ddd, J=17.4, 13.4, 5.4 Hz, 1H), 2.64-2.55 (m, 1H), 2.39 (td, J=13.2, 4.5 Hz, 1H), 1.97 (d, J=12.8 Hz, 1H), 1.38 (dd, J=14.2, 9.4 Hz, 1H), 1.13 (d, J=13.8 Hz, 1H), 0.94 (s, 9H).

### Example 87: Preparation of (2S,3S,4S,5R)-4-(3-chloro-2-fluorophenyl)-N-(4-(4-(4-(2-(2,6-dioxopiperidin-3-yl)-1-oxoisoindolin-5-yl)piperazin-1-yl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

### Synthesis of tert-butyl 4-(4-(2-(2,6-dioxopiperidin-3-yl)-1-oxoisoindolin-5-yl)piperazin-1-yl)piperidine-1-carboxylate

Into a 100 mL round-bottom flask were added 3-(5-iodo-1-oxoisoindolin-2-yl)piperidine-2,6-dione (1.5 g, 4.0 mmol, 1.0 eq), tert-butyl 4-(piperazin-1-yl)piperidine-1-carboxylate (1.2 g, 4.4 mmol, 1.1 eq), Ruphos Pd G3 (339.3 mg, 0.4 mmol, 0.1 eq), Cs₂CO₃ (4.0 g, 12.0 mmol, 3.0 eq) and DMF (30 mL). The resulting mixture was stirred for 16 hours at 100° C. under nitrogen atmosphere. The mixture was diluted with water (100 mL) and then extracted with CH₂Cl₂ (2×100 mL). The combined organic layers were washed with brine (100 mL), dried over anhydrous Na₂SO₄. After filtration, the filtrate was concentrated under reduced pressure. The residue was purified by silica gel column (dichloromethane/methanol=100:1-10:1) to afford tert-butyl 4-(4-(2-(2,6-dioxopiperidin-3-yl)-1-oxoisoindolin-5-yl)piperazin-1-yl)piperidine-1-carboxylate (502.8 mg, 25.0%) as a brown solid. LC-MS: (ESI, m/z): M+1: 512.

### Synthesis of 3-(1-oxo-5-(4-(piperidin-4-yl)piperazin-1-yl)isoindolin-2-yl)piperidine-2,6-dione

Into a 50 mL round-bottom flask were added tert-butyl 4-(4-(2-(2,6-dioxopiperidin-3-yl)-1-oxoisoindolin-5-yl)piperazin-1-yl)piperidine-1-carboxylate (465 mg, 1.1 mmol, 1.0 eq) and HCl/dioxane (5 mL, 4 M). The mixture was stirred for 3 hours at 25° C. The resulting mixture was concentrated under reduced pressure to afford 3-(1-oxo-5-(4-(piperidin-4-yl)piperazin-1-yl)isoindolin-2-yl)piperidine-2,6-dione (497 mg, 96%) as a white solid. LC-MS: (ESI, m/z): M+1: 412.

### Synthesis of 3-(5-(4-(1-(3-methoxy-4-nitrobenzoyl)piperidin-4-yl)piperazin-1-yl)-1-oxoisoindolin-2-yl)piperidine-2,6-dione

Into a 25 mL round-bottom flask were added 3-(1-oxo-5-(4-(piperidin-4-yl)piperazin-1-yl)isoindolin-2-yl)piperidine-2,6-dione (447.5 mg, 1.0 mmol, 1.0 eq), 3-methoxy-4-nitrobenzoic acid (177.3 mg, 0.9 mmol, 0.9 eq), HATU (512.8 mg, 1.3 mmol, 1.3 eq), DIEA (522.9 mg, 4.0 mmol, 4.0 eq) and DMF (5 mL). The resulting mixture was stirred for 2 hours at 25° C. The reaction mixture was diluted with water (25 mL) and extracted with CH₂Cl₂ (2×25 mL). The combined organic layers were washed with brine (30 mL), dried over anhydrous Na₂SO₄. After filtration, the filtrate was concentrated under reduced pressure. The crude residue was purified by silica gel column (dichloromethane/methanol=100:1-30:1) to afford 3-(5-(4-(1-(3-methoxy-4-nitrobenzoyl)piperidin-4-yl)piperazin-1-yl)-1-oxoisoindolin-2-yl)piperidine-2,6-dione (192 mg, 30%) as a yellow solid. LC-MS: (ESI, m/z): M+1: 591.

### Synthesis of 3-(5-(4-(1-(4-amino-3-methoxybenzoyl)piperidin-4-yl)piperazin-1-yl)-1-oxoisoindolin-2-yl)piperidine-2,6-dione

Into a 10 mL round-bottom flask were added 2-(2,6-dioxopiperidin-3-yl)-5-(4-(3-methoxy-4-nitrobenzoyl)piperazin-1-yl)isoindoline-1,3-dione (172.0 mg, 0.3 mmol, 1.0 eq), Fe (81.0 mg, 1.5 mmol, 5.0 eq), NH₄Cl (155.0 mg, 3.0 mmol, 10.0 eq), water (2.0 mL) and EtOH (4.0 mL) at 25° C. The resulting mixture was stirred for 16 hours at 75° C. The reaction mixture was filtered, and the filter cake was washed with EtOH (8×5 mL). The combined filtrate was collected and concentrated. The residue was washed with water (2×5 mL), and then dried under vacuum to afford 3-(5-(4-(1-(4-amino-3-methoxybenzoyl)piperidin-4-yl)piperazin-1-yl)-1-oxoisoindolin-2-yl)piperidine-2,6-dione (85.0 mg, 52.1%) as a yellow solid. LC-MS: (ESI, m/z): M+1: 561.

### Synthesis of (2S,3S,4S,5R)-4-(3-chloro-2-fluorophenyl)-N-(4-(4-(4-(2-(2,6-dioxopiperidin-3-yl)-1-oxoisoindolin-5-yl)piperazin-1-yl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

To a solution of 3-(5-(4-(1-(4-amino-3-methoxybenzoyl)piperidin-4-yl)piperazin-1-yl)-1-oxoisoindolin-2-yl)piperidine-2,6-dione (75.0 mg, 0.13 mmol, 1.0 eq) in MeCN (3 mL) were added (2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxylic acid (97.5 mg, 0.2 mmol, 1.5 eq), TCFH (112.6 mg, 0.4 mmol, 3.0 eq) and NMI (98.9 mg, 1.2 mmol, 9.0 eq). The resulting mixture was stirred for 2 hours at 25° C. The reaction was quenched with water (10 mL) and extracted with CHCl₃ (2×10 mL). The combined organic layers were washed with brine (10 mL), dried over anhydrous Na₂SO₄. After filtration, the filtrate was concentrated under reduced pressure. The residue was purified by silica gel column chromatography, eluted with dichloromethane/methanol=100:1-30:1 and then re-purified by Prep-HPLC using the following conditions: Column: XBridge C18 OBD Column, 19*150 mm, 5 μm; Mobile Phase A: Water (0.1% FA), Mobile Phase B: CH₃CN; Flow rate: 20 mL/min; Gradient: 5% B to 65% B in 9 min, 65% B; Wave Length: 254/220 nm; RT=8.2 mins. Finally, (2S,3S,4S,5R)-4-(3-chloro-2-fluorophenyl)-N-(4-(4-(4-(2-(2,6-dioxopiperidin-3-yl)-1-oxoisoindolin-5-yl)piperazin-1-yl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide (12.11 mg, 8.8%) was obtained as a white solid. LC-MS: (ESI, m/z): M/2+1: 515. ¹HNMR (500 MHz, Methanol-d4) δ 8.29-8.27 (d, J=10.0 Hz, 1H), 8.16 (s, 1H), 7.56-7.55 (d, J=5.0 Hz, 1H), 7.25-7.23 (m, 2H), 7.07-6.98 (m, 4H), 6.92 (d, J=5.0 Hz, 1H), 6.57 (s, 1H), 5.01-4.99 (m, 1H)), 4.37-4.35 (m, 1H), 4.31-4.30 (m, 1H), 4.19 (m, 1H), 3.85 (s, 3H), 3.69-3.66 (m, 1H), 3.49 (m, 1H), 3.44 (m, 1H), 2.79-2.61 (m, 7H), 2.35-2.34 (m, 2H), 2.06-2.03 (m, 3H), 1.45-1.35 (m, 2H), 1.32-1.26 (m, 2H), 0.92 (s, 9H).

### Example 88: Preparation of (2S,3S,4S,5R)-4-(3-chloro-2-fluorophenyl)-N-(4-(9-(2-(2,6-dioxopiperidin-3-yl)-1-oxoisoindolin-5-yl)-3,9-diazaspiro[5.5]undecane-3-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

### Synthesis of tert-butyl 9-(2-(2,6-dioxopiperidin-3-yl)-1-oxoisoindolin-5-yl)-3,9-diazaspiro[5.5]undecane-3-carboxylate

Into a 50 mL round-bottom flask were added 3-(5-iodo-1-oxoisoindolin-2-yl)piperidine-2,6-dione (628.0 mg, 1.7 mmol, 1.0 eq), tert-butyl 3,9-diazaspiro[5.5]undecane-3-carboxylate (432 mg, 1.7 mmol, 1.0 eq), Ruphos Pd G3 (141.0 mg, 0.17 mmol, 0.1 eq), Cs₂CO₃ (1.6 g, 5.1 mmol, 3.0 eq) and DMF (6 mL). The mixture was stirred for 16 hours at 100° C. under nitrogen atmosphere. The reaction mixture was diluted with water (30 mL) and then extracted with CH₂Cl₂ (2×30 mL). The combined organic layers were washed with brine (30 mL), dried over anhydrous Na₂SO₄. After filtration, the filtrate was concentrated under reduced pressure. The residue was purified by silica gel column (dichloromethane/methanol=100:1-10:1) to afford tert-butyl 9-(2-(2,6-dioxopiperidin-3-yl)-1-oxoisoindolin-5-yl)-3,9-diazaspiro[5.5]undecane-3-carboxylate (109.0 mg, 12.9%) as a brown solid. LC-MS: (ESI, m/z): M+1: 497.

### Synthesis of 3-(1-oxo-5-(3,9-diazaspiro[5.5]undecan-3-yl)isoindolin-2-yl)piperidine-2,6-dione

Into a 10 mL round-bottom flask were added tert-butyl 9-(2-(2,6-dioxopiperidin-3-yl)-1-oxoisoindolin-5-yl)-3,9-diazaspiro[5.5]undecane-3-carboxylate (109.0 mg, 0.22 mmol, 1.0 eq) and HCl/dioxane (1 mL, 4 M). The resulting mixture was stirred for 3 hours at 25° C. The reaction mixture was concentrated under reduced pressure to afford 3-(1-oxo-5-(3,9-diazaspiro[5.5]undecan-3-yl)isoindolin-2-yl)piperidine-2,6-dione (95 mg, crude) as a white solid. LC-MS: (ESI, m/z): M+1: 397.

### Synthesis of 3-(5-(9-(3-methoxy-4-nitrobenzoyl)-3,9-diazaspiro[5.5]undecan-3-yl)-1-oxoisoindolin-2-yl)piperidine-2,6-dione

Into a 25 mL round-bottom flask were added 3-(1-oxo-5-(3,9-diazaspiro[5.5]undecan-3-yl)isoindolin-2-yl)piperidine-2,6-dione (95.0 mg, 0.22 mmol, 1.0 eq), 3-methoxy-4-nitrobenzoic acid (47.0 mg, 0.24 mmol, 1.1 eq), HATU (124.0 mg, 0.33 mmol, 1.5 eq), DIEA (99.0 mg, 0.77 mmol, 3.5 eq) and DMF (1 mL). The resulting mixture was stirred for 2 hours at 25° C. The reaction mixture was diluted with water (5 mL) and then extracted with CH₂Cl₂ (2×5 mL). The combined organic layers were washed with brine (10 mL), dried over anhydrous Na₂SO₄. After filtration, the filtrate was concentrated under reduced pressure. The crude residue was purified by silica gel column (dichloromethane/methanol=100:1-20:1) to afford 3-(5-(9-(3-methoxy-4-nitrobenzoyl)-3,9-diazaspiro[5.5]undecan-3-yl)-1-oxoisoindolin-2-yl)piperidine-2,6-dione (42 mg, 30%) as a yellow solid. LC-MS: (ESI, m/z): M+1: 576.

### Synthesis of 3-(5-(9-(4-amino-3-methoxybenzoyl)-3,9-diazaspiro[5.5]undecan-3-yl)-1-oxoisoindolin-2-yl)piperidine-2,6-dione

Into a 10 mL round-bottom flask were added 2-(2,6-dioxopiperidin-3-yl)-5-(4-(3-methoxy-4-nitrobenzoyl)piperazin-1-yl)isoindoline-1,3-dione (42.0 mg, 0.07 mmol, 1.0 eq), Fe (20.0 mg, 0.35 mmol, 5.0 eq), NH₄Cl (39.0 mg, 0.7 mmol, 10.0 eq), water (0.4 mL) and EtOH (0.8 mL). The resulting mixture was stirred for 16 hours at 75° C. The reaction mixture was filtered, and the filter cake was washed with EtOH (8×5 mL). The combined filtrate was collected and concentrated. The residue was washed with water (2×5 mL), and then dried under vacuum to afford 3-(5-(9-(4-amino-3-methoxybenzoyl)-3,9-diazaspiro[5.5]undecan-3-yl)-1-oxoisoindolin-2-yl)piperidine-2,6-dione (24.0 mg, 61.0%) as a yellow solid. LC-MS: (ESI, m/z): M+1: 546.

### Synthesis of (2S,3S,4S,5R)-4-(3-chloro-2-fluorophenyl)-N-(4-(9-(2-(2,6-dioxopiperidin-3-yl)-1-oxoisoindolin-5-yl)-3,9-diazaspiro[5.5]undecane-3-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

To a solution of 3-(5-(9-(4-amino-3-methoxybenzoyl)-3,9-diazaspiro[5.5]undecan-3-yl)-1-oxoisoindolin-2-yl)piperidine-2,6-dione (24.0 mg, 0.044 mmol, 1.0 eq) in MeCN (1 mL) were added (2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxylic acid (32.0 mg, 0.066 mmol, 1.5 eq), TCFH (37.0 mg, 0.132 mmol, 3.0 eq) and NMI (32.0 mg, 0.395 mmol, 9.0 eq). The resulting mixture was stirred for 2 hours at 25° C. The reaction was quenched with water (5 mL) and extracted with CHCl₃ (2×5 mL). The combined organic layers were washed with brine (5 mL), dried over anhydrous Na₂SO₄. After filtration, the filtrate was concentrated under reduced pressure. The residue was purified by silica gel column chromatography, eluted with dichloromethane/methanol=100:1-20:1 and then re-purified by Prep-HPLC using the following conditions: Column: XBridge C18 OBD Column, 19*150 mm, 5 μm; Mobile Phase A: Water (0.1% FA), Mobile Phase B: CH₃CN; Flow rate: 20 mL/min; Gradient: 5% B to 60% B in 9 min, 60% B; Wave Length: 254/220 nm; RT=8.4 mins. Finally, (2S,3S,4S,5R)-4-(3-chloro-2-fluorophenyl)-N-(4-(9-(2-(2,6-dioxopiperidin-3-yl)-1-oxoisoindolin-5-yl)-3,9-diazaspiro[5.5]undecane-3-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide (2.12 mg, 4.8%) was obtained as a white solid. LC-MS: (ESI, m/z): M/2+1: 507. ¹HNMR (500 MHz, Methanol-d4) δ 8.42 (s, 1H), 8.27-8.26 (d, J=5.0 Hz, 1H), 7.54-7.52 (d, J=10.0 Hz, 1H), 7.30-7.25 (m, 2H), 7.07-7.06 (m, 1H), 7.01 (m, 1H), 6.97 (m, 2H), 6.92 (m, 1H), 6.56 (s, 1H), 5.01-5.00 (m, 1H)), 4.37-4.35 (m, 1H), 4.31 (m, 1H), 4.29 (m, 1H), 3.86 (s, 3H), 3.69-3.66 (m, 2H), 3.49 (m, 2H), 3.47 (m, 1H) 3.44 (m, 4H), 2.80-2.77 (m, 1H), 2.70 (m, 1H), 2.38-2.35 (m, 1H), 2.10 (m, 1H), 1.64 (m, 8H), 1.34 (m, 1H), 1.26-1.21 (m, 4H), 0.92 (s, 9H).

### Example 89: Preparation of (2S,3S,4S,5R)-4-(3-chloro-2-fluorophenyl)-N-(4-(4-((2-(2,6-dioxopiperidin-3-yl)-1-oxoisoindolin-5-yl)methyl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

### Synthesis of 3-(1-oxo-5-vinylisoindolin-2-yl)piperidine-2,6-dione

Into a 50 mL round-bottom flask were added 3-(5-bromo-1-oxoisoindolin-2-yl)piperidine-2,6-dione (2.0 g, 6.2 mmol, 1.0 eq), tributyl(vinyl)stannane (2.9 g, 9.2 mmol, 1.5 eq), Pd(PPh₃)₂Cl₂ (0.4 g, 0.6 mmol, 0.1 eq) and dioxane (40 mL). The mixture was stirred for 16 hours at 110° C. under nitrogen atmosphere. The resulting mixture was concentrated under reduced pressure. The residue was slurried with MTBE (2×40 mL) and then filtered. The filter cake was collected and dried under vacuum to afford 3-(1-oxo-5-vinylisoindolin-2-yl)piperidine-2,6-dione (900.0 mg, 53.0%) as a brown solid. ¹HNMR (500 MHz, DMSO-d₆) δ 10.96 (s, 1H), 7.71-7.70 (d, J=5.0 Hz, 1H), 7.63-7.61 (d, J=10.0 Hz, 1H), 6.90-6.84 (m, 1H)), 6.01-5.98 (d, J=15.0 Hz, 1H), 5.42-5.40 (d, J=10.0 Hz, 1H), 5.12-5.09 (m, 1H), 4.48-4.32 (m, 2H), 2.94-2.89 (m, 1H), 2.62-2.59 (m, 1H), 2.42-2.40 (m, 1H), 2.02-2.00 (m, 1H).

### Synthesis of 2-(2,6-dioxopiperidin-3-yl)-1-oxoisoindoline-5-carbaldehyde

Into a 100 mL round-bottom flask were added 3-(1-oxo-5-vinylisoindolin-2-yl)piperidine-2,6-dione (0.85 g, 3.14 mmol, 1.0 eq), NaIO₄ (1.35 g, 6.28 mmol, 2.0 eq), K₂OsO₄·2H₂O (0.14 g, 0.31 mmol, 0.1 eq), 2,6-Lutidine (0.67 g, 6.28 mmol, 2.0 eq), dioxane (17 mL) and water (3.4 mL). The mixture was stirred for 16 hours at 25° C. The resulting mixture was diluted with water (50 mL) and then extracted with CH₂Cl₂ (2×50 mL). The combined organic layers were washed with brine (50 mL), dried over anhydrous Na₂SO₄. After filtration, the filtrate was concentrated under reduced pressure. The crude residue was purified by silica gel column (dichloromethane/methanol=100:1-20:1) to afford 2-(2,6-dioxopiperidin-3-yl)-1-oxoisoindoline-5-carbaldehyde (490 mg, 54%) as a white solid. ¹HNMR (500 MHz, DMSO-d₆) δ 11.00 (s, 1H), 10.15 (s, 1H), 8.15 (s, 1H), 8.07-8.05 (d, J=10.0 Hz, 1H), 7.95-7.93 (d, J=10.0 Hz, 1H), 5.17-5.14 (m, 1H), 4.60-4.44 (m, 2H), 2.95-2.88 (m, 1H), 2.63-2.60 (m, 1H), 2.43-2.41 (m, 1H), 2.06 (m, 1H).

### Synthesis of tert-butyl 4-((2-(2,6-dioxopiperidin-3-yl)-1-oxoisoindolin-5-yl)methyl)piperazine-1-carboxylate

Into a 25 mL round-bottom flask were added 2-(2,6-dioxopiperidin-3-yl)-1-oxoisoindoline-5-carbaldehyde (490.0 mg, 1.8 mmol, 1.0 eq), tert-butyl piperazine-1-carboxylate (369.0 mg, 2.0 mmol, 1.1 eq), AcOH (0.5 mL) and DCM (10 mL). The resulting mixture was stirred for 3 hours at 25° C. After that, NaBH(OAc)₃ (763.0 mg, 3.6 mmol, 2.0 eq) was added in portions. The reaction mixture was stirred for 12 hours at 25° C. The resulting mixture was diluted with water (30 mL) and extracted with CH₂Cl₂ (2×30 mL). The combined organic layers were washed with brine (30 mL), dried over anhydrous Na₂SO₄. After filtration, the filtrate was concentrated under reduced pressure. The crude residue was purified by silica gel column (dichloromethane/methanol=100:1-20:1) to afford tert-butyl 4-((2-(2,6-dioxopiperidin-3-yl)-1-oxoisoindolin-5-yl)methyl)piperazine-1-carboxylate (260.0 mg, 32%) as a yellow solid. LC-MS: (ESI, m/z): M+1: 443.

### Synthesis of 3-(1-oxo-5-(piperazin-1-ylmethyl)isoindolin-2-yl)piperidine-2,6-dione

Into a 10 mL round-bottom flask were added tert-butyl 4-((2-(2,6-dioxopiperidin-3-yl)-1-oxoisoindolin-5-yl)methyl)piperazine-1-carboxylate (260.0 mg, 0.58 mmol, 1.0 eq) and HCl/dioxane (2 mL, 4 M). The resulting mixture was stirred for 3 hours at 25° C. The reaction mixture was concentrated under reduced pressure to afford 3-(1-oxo-5-(piperazin-1-ylmethyl)isoindolin-2-yl)piperidine-2,6-dione (244.0 mg, crude) as a white solid. LC-MS: (ESI, m/z): M+1: 343.

### Synthesis of 3-(5-((4-(3-methoxy-4-nitrobenzoyl)piperazin-1-yl)methyl)-1-oxoisoindolin-2-yl)piperidine-2,6-dione

Into a 25 mL round-bottom flask were added 3-(1-oxo-5-(piperazin-1-ylmethyl)isoindolin-2-yl)piperidine-2,6-dione (244.0 mg, 0.58 mmol, 1.0 eq), 3-methoxy-4-nitrobenzoic acid (115.0 mg, 0.58 mmol, 1.0 eq), HATU (330.0 mg, 0.87 mmol, 1.5 eq), DIEA (300.0 mg, 2.32 mmol, 4.0 eq) and DMF (2.5 mL). The resulting mixture was stirred for 2 hours at 25° C. The reaction mixture was diluted with water (10 mL) and then extracted with CH₂Cl₂ (2×10 mL). The combined organic layers were washed with brine (10 mL), and dried over anhydrous Na₂SO₄. After filtration, the filtrate was concentrated under reduced pressure. The crude residue was purified by silica gel column (dichloromethane/methanol=100:1-20:1) to afford 3-(5-((4-(3-methoxy-4-nitrobenzoyl)piperazin-1-yl)methyl)-1-oxoisoindolin-2-yl)piperidine-2,6-dione (174.0 mg, 46.0%) as a yellow solid. LC-MS: (ESI, m/z): M+1: 522.

### Synthesis of 3-(5-((4-(4-amino-3-methoxybenzoyl)piperazin-1-yl)methyl)-1-oxoisoindolin-2-yl)piperidine-2,6-dione

Into a 10 mL round-bottom flask were added 3-(5-((4-(3-methoxy-4-nitrobenzoyl)piperazin-1-yl)methyl)-1-oxoisoindolin-2-yl)piperidine-2,6-dione (174.0 mg, 0.33 mmol, 1.0 eq), Fe (93.0 mg, 1.67 mmol, 5.0 eq), NH₄Cl (178.0 mg, 3.34 mmol, 10.0 eq), water (1.7 mL) and EtOH (3.5 mL). The resulting mixture was stirred for 16 hours at 75° C. The reaction mixture was filtered, and the filter cake was washed with EtOH (8×5 mL). The combined filtrate was collected and concentrated. The residue was washed with water (2×5 mL), and then dried under vacuum to afford 3-(5-((4-(4-amino-3-methoxybenzoyl)piperazin-1-yl)methyl)-1-oxoisoindolin-2-yl)piperidine-2,6-dione (163.0 mg, 49.0%) as a yellow solid. ¹HNMR (500 MHz, DMSO-d₆) δ 10.96 (s, 1H), 7.70 (m, 1H), 7.57 (m, 1H), 7.48 (m, 1H), 6.84-6.78 (m, 2H), 6.62-6.60 (d, J=10.0 Hz, 1H), 5.11-5.09 (m, 2H), 4.47-4.31 (m, 2H), 3.77 (s, 3H), 3.63-3.51 (m, 5H), 2.95-2.87 (m, 1H), 2.62 (m, 1H), 2.41-2.36 (m, 4H), 2.02-1.99 (m, 1H), 1.25-1.24 (m, 1H).

### Synthesis of (2S,3S,4S,5R)-4-(3-chloro-2-fluorophenyl)-N-(4-(4-((2-(2,6-dioxopiperidin-3-yl)-1-oxoisoindolin-5-yl)methyl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

To a solution of 3-(5-((4-(4-amino-3-methoxybenzoyl)piperazin-1-yl)methyl)-1-oxoisoindolin-2-yl)piperidine-2,6-dione (60.0 mg, 0.12 mmol, 1.0 eq) in MeCN (1.8 mL) were added (2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxylic acid (94.0 mg, 0.19 mmol, 1.5 eq), TCFH (68.0 mg, 0.24 mmol, 2.0 eq) and NMI (33.0 mg, 0.40 mmol, 3.3 eq). The resulting mixture was stirred for 2 hours at 25° C. The reaction was quenched with water (5 mL) and extracted with CHCl₃ (2×5 mL). The combined organic layers were washed with brine (5 mL), and dried over anhydrous Na₂SO₄. After filtration, the filtrate was concentrated under reduced pressure. The residue was purified by silica gel column chromatography, eluted with dichloromethane/methanol=100:1-20:1 and then re-purified by Prep-HPLC using the following conditions: Column: XBridge C18 OBD Column, 19*150 mm, 5 μm; Mobile Phase A: Water (0.1% FA), Mobile Phase B: CH₃CN; Flow rate: 20 mL/min; Gradient: 5% B to 55% B in 9 min, 55% B; Wave Length: 254/220 nm; RT=8.7 mins. Finally, (2S,3S,4S,5R)-4-(3-chloro-2-fluorophenyl)-N-(4-(4-((2-(2,6-dioxopiperidin-3-yl)-1-oxoisoindolin-5-yl)methyl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide (2.02 mg, 1.7%) was obtained as a white solid. LC-MS: (ESI, m/z): M/2+1: 480. ¹HNMR (500 MHz, Methanol-d₄) δ 8.35-8.33 (d, J=10.0 Hz, 1H), 8.23 (s, 1H), 7.77-7.75 (d, J=10.0 Hz, 1H), 7.59 (s, 1H), 7.52 (m, 1H), 7.33 (m, 2H), 7.14 (m, 1H), 7.09 (s, 1H), 6.98-6.97 (d, J=5.0 Hz, 1H), 6.95 (s, 1H), 5.16-5.12 (m, 1H)), 4.52-4.43 (m, 1H), 4.27 (m, 1H), 3.93 (s, 3H), 3.74 (m, 2H), 3.69 (m, 3H), 3.57 (m, 3H) 3.46 (m, 1H), 2.79 (m, 1H), 2.55 (m, 1H), 2.53 (m, 1H), 2.50-2.47 (m, 5H), 2.14 (m, 1H), 1.42 (m, 1H), 1.34 (m, 3H), 1.00 (s, 9H).

### Example 90: Preparation of (2S,3S,4S,5R)-4-(3-chloro-2-fluorophenyl)-N-(4-(4-((4-(2-(2,6-dioxopiperidin-3-yl)-1-oxoisoindolin-5-yl)piperazin-1-yl)methyl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

### Synthesis of tert-butyl 4-(2-(2,6-dioxopiperidin-3-yl)-1-oxoisoindolin-5-yl)piperazine-1-carboxylate

Into a 50 mL round-bottom flask were added 3-(5-iodo-1-oxoisoindolin-2-yl)piperidine-2,6-dione (628.0 mg, 1.7 mmol, 1.0 eq), tert-butyl piperazine-1-carboxylate (316.6 mg, 1.7 mmol, 1.0 eq), Ruphos Pd G3 (141.0 mg, 0.17 mmol, 0.1 eq), Cs₂CO₃ (1.6 g, 5.1 mmol, 3.0 eq) and DMF (6 mL). The resulting mixture was stirred for 16 hours at 100° C. under nitrogen atmosphere. The reaction mixture was diluted with water (30 mL) and then extracted with CH₂Cl₂ (2×30 mL). The combined organic layers were washed with brine (30 mL), dried over anhydrous Na₂SO₄. After filtration, the filtrate was concentrated under reduced pressure. The residue was purified by silica gel column (dichloromethane/methanol=100:1-10:1) to afford tert-butyl 4-(2-(2,6-dioxopiperidin-3-yl)-1-oxoisoindolin-5-yl)piperazine-1-carboxylate (203 mg, 28%) as a brown solid. LC-MS: (ESI, m/z): M+1: 429.

### Synthesis of 3-(1-oxo-5-(piperazin-1-yl)isoindolin-2-yl)piperidine-2,6-dione

Into a 10 mL round-bottom flask were added tert-butyl 4-(2-(2,6-dioxopiperidin-3-yl)-1-oxoisoindolin-5-yl)piperazine-1-carboxylate (203.0 mg, 0.47 mmol, 1.0 eq) and HCl/dioxane (2 mL, 4 M). The mixture was stirred for 3 hours at 25° C. The reaction mixture was concentrated under reduced pressure to afford 3-(1-oxo-5-(piperazin-1-yl)isoindolin-2-yl)piperidine-2,6-dione (210 mg, crude) as a white solid. LC-MS: (ESI, m/z): M+1: 329.

### Synthesis of tert-butyl 4-((4-(2-(2,6-dioxopiperidin-3-yl)-1-oxoisoindolin-5-yl)piperazin-1-yl)methyl)piperidine-1-carboxylate

Into a 25 mL round-bottom flask were added 3-(1-oxo-5-(piperazin-1-yl)isoindolin-2-yl)piperidine-2,6-dione (210.0 mg, 0.57 mmol, 1.0 eq), tert-butyl 4-formylpiperidine-1-carboxylate (133.0 mg, 0.62 mmol, 1.1 eq), AcOH (0.2 mL) and DCM (4 mL). The resulting mixture was stirred for 3 hours at 25° C. After that, NaBH(OAc)₃ (241.0 mg, 1.1 mmol, 2.0 eq) was added in portions. The resulting mixture was stirred for 12 hours. The reaction was diluted with water (10 mL) and extracted with CH₂Cl₂ (2×10 mL). The combined organic layers were washed with brine (10 mL), dried over anhydrous Na₂SO₄. After filtration, the filtrate was concentrated under reduced pressure. The crude residue was purified by silica gel column (dichloromethane/methanol=100:1-20:1) to afford tert-butyl 4-((4-(2-(2,6-dioxopiperidin-3-yl)-1-oxoisoindolin-5-yl)piperazin-1-yl)methyl)piperidine-1-carboxylate (270 mg, 87%) as a yellow solid. LC-MS: (ESI, m/z): M+1: 526.

### Synthesis of 3-(1-oxo-5-(4-(piperidin-4-ylmethyl)piperazin-1-yl)isoindolin-2-yl)piperidine-2,6-dione

Into a 10 mL round-bottom flask were added tert-butyl 4-((4-(2-(2,6-dioxopiperidin-3-yl)-1-oxoisoindolin-5-yl)piperazin-1-yl)methyl)piperidine-1-carboxylate (270.0 mg, 0.51 mmol, 1.0 eq) and HCl/dioxane (2 mL, 4 M). The resulting mixture was stirred for 3 hours at 25° C. The reaction mixture was concentrated under reduced pressure to afford 3-(1-oxo-5-(4-(piperidin-4-ylmethyl)piperazin-1-yl)isoindolin-2-yl)piperidine-2,6-dione (300 mg, crude) as a white solid. LC-MS: (ESI, m/z): M+1: 426.

### Synthesis of 3-(5-(4-((1-(3-methoxy-4-nitrobenzoyl)piperidin-4-yl)methyl)piperazin-1-yl)-1-oxoisoindolin-2-yl)piperidine-2,6-dione

Into a 25 mL round-bottom flask were added 3-(1-oxo-5-(4-(piperidin-4-ylmethyl)piperazin-1-yl)isoindolin-2-yl)piperidine-2,6-dione (300.0 mg, 0.9 mmol, 1.0 eq), 3-methoxy-4-nitrobenzoic acid (88.7 mg, 0.8 mmol, 0.9 eq), HATU (285.5 mg, 1.4 mmol, 1.5 eq), DIEA (465.5 mg, 3.6 mmol, 4.0 eq) and DMF (3 mL). The resulting mixture was stirred for 2 hours at 25° C. The reaction mixture was diluted with water (10 mL) and then extracted with CH₂Cl₂ (2×10 mL). The combined organic layers were washed with brine (10 mL), dried over anhydrous Na₂SO₄. After filtration, the filtrate was concentrated under reduced pressure. The crude residue was purified by silica gel column (dichloromethane/methanol=100:1-20:1) to afford 3-(5-(4-((1-(3-methoxy-4-nitrobenzoyl)piperidin-4-yl)methyl)piperazin-1-yl)-1-oxoisoindolin-2-yl)piperidine-2,6-dione (79.5 mg, 21.7%) as yellow solid. LC-MS: (ESI, m/z): M+1: 605.

### Synthesis of 3-(5-(4-((1-(4-amino-3-methoxybenzoyl)piperidin-4-yl)methyl)piperazin-1-yl)-1-oxoisoindolin-2-yl)piperidine-2,6-dione

Into a 10 mL round-bottom flask were added 3-(5-(4-((1-(3-methoxy-4-nitrobenzoyl)piperidin-4-yl)methyl)piperazin-1-yl)-1-oxoisoindolin-2-yl)piperidine-2,6-dione (79.5 mg, 0.13 mmol, 1.0 eq), Fe (36.7 mg, 0.66 mmol, 5.0 eq), NH₄Cl (70.3 mg, 1.31 mmol, 10.0 eq), water (0.8 mL) and EtOH (1.6 mL). The resulting mixture was stirred for 16 hours at 75° C. The reaction mixture was filtered. The filter cake was washed with EtOH (8×5 mL). The combined filtrate was collected and concentrated. The residue was washed with water (2×5 mL), and then dried under vacuum to afford 3-(5-(4-((1-(4-amino-3-methoxybenzoyl)piperidin-4-yl)methyl)piperazin-1-yl)-1-oxoisoindolin-2-yl)piperidine-2,6-dione (33 mg, 44%) as a yellow solid. ¹HNMR (500 MHz, CD₃OD) δ 7.88-7.86 (d, J=10.0 Hz, 1H), 7.66-7.63 (m, 1H), 7.29 (s, 1H), 7.09-7.07 (m, 3H), 5.34 (m, 2H), 5.11 (m, 2H), 4.44 (m, 4H), 3.98 (s, 3H), 3.67-3.64 (m, 5H), 2.90 (m, 3H), 2.19 (m, 1H), 2.15 (m, 3H), 2.08 (m, 3H), 2.04 (m, 2H), 2.02 (m, 2H).

### Synthesis of (2S,3S,4S,5R)-4-(3-chloro-2-fluorophenyl)-N-(4-(4-((4-(2-(2,6-dioxopiperidin-3-yl)-1-oxoisoindolin-5-yl)piperazin-1-yl)methyl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

To a solution of 3-(5-(4-((1-(4-amino-3-methoxybenzoyl)piperidin-4-yl)methyl)piperazin-1-yl)-1-oxoisoindolin-2-yl)piperidine-2,6-dione (33.0 mg, 0.06 mmol, 1.0 eq) in MeCN (1.0 mL) were added (2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxylic acid (35.5 mg, 0.07 mmol, 1.3 eq), TCFH (32.2 mg, 0.11 mmol, 2.0 eq) and NMI (15.6 mg, 0.19 mmol, 3.3 eq). The resulting mixture was stirred for 2 hours at 25° C. The reaction was quenched with water (5 mL) and extracted with CHCl₃ (2×5 mL). The combined organic layers were washed with brine (5 mL), dried over anhydrous Na₂SO₄. After filtration, the filtrate was concentrated under reduced pressure. The residue was purified by silica gel column chromatography, eluted with dichloromethane/methanol=100:1-20:1 and then re-purified by Prep-HPLC using the following conditions: Column: XBridge C18 OBD Column, 19*150 mm, 5 μm; Mobile Phase A: Water (0.1% FA), Mobile Phase B: CH₃CN; Flow rate: 20 mL/min; Gradient: 5% B to 65% B in 9 min, 65% B; Wave Length: 254/220 nm; RT=8.3 mins. Finally, (2S,3S,4S,5R)-4-(3-chloro-2-fluorophenyl)-N-(4-(4-((4-(2-(2,6-dioxopiperidin-3-yl)-1-oxoisoindolin-5-yl)piperazin-1-yl)methyl)piperidine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide (1.28 mg, 2.1%) was obtained as a white solid. LC-MS: (ESI, m/z): M/2+1: 522. ¹HNMR (500 MHz, Methanol-d4) δ 8.36-8.34 (d, J=10.0 Hz, 1H), 8.31 (s, 1H), 8.24 (s, 1H), 7.98 (s, 1H), 7.65-7.64 (d, J=5.0 Hz, 1H), 7.39-7.34 (m, 1H), 7.15 (m, 1H), 7.09 (m, 1H), 6.99-6.98 (d, J=5.0 Hz, 1H), 6.65 (s, 1H), 5.35-5.34 (m, 1H)), 5.11-5.10 (m, 1H), 4.53 (m, 1H), 4.47 (m, 3H), 4.41 (m, 1H), 3.94 (s, 3H), 3.75 (m, 1H) 3.56 (m, 1H), 3.45 (m, 1H), 3.41 (m, 4H), 2.76 (m, 5H), 2.47-2.45 (m, 3H), 2.19-2.18 (m, 2H), 2.03-2.02 (m, 2H), 1.94 (m, 1H), 1.46 (m, 1H), 1.32 (m, 6H), 1.01 (s, 9H).

### Example 91: Preparation of (2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-isopropoxyphenyl)-2-neopentyl-6′-(trifluoromethyl)-1′,2′-dihydrospiro[pyrrolidine-3,3′-pyrrolo[3,2-c]pyridine]-5-carboxamide

### Synthesis of 3-isopropoxy-4-nitrobenzoic acid

To a solution of propan-2-ol (324.0 mg, 5.4 mmol, 1.0 eq) in THF (15 mL) was added NaH (432.0 mg, 10.8 mmol, 2.0 eq., 60% in mineral oil) in portions at 0° C. The resulting mixture was stirred for 0.5 hour at 0° C. and then a solution of 3-fluoro-4-nitrobenzoic acid (1.0 g, 5.4 mmol, 1.0 eq) in THF (5 mL) was added at 0° C. The reaction was warmed up to 25° C. and stirred for 12 hours. The reaction was quenched with sat. NH₄Cl (30 mL) at 0° C. and extracted with CHCl₃ (2×50 mL). The combined organic layers were washed with brine (50 mL), dried over anhydrous Na₂SO₄. After filtration, the filtrate was concentrated under reduced pressure to afford 3-isopropoxy-4-nitrobenzoic acid (1.3 g, 100%) as yellow solid. ¹HNMR (500 MHz, Methanol-d₄) δ 7.81 (s, 1H), 7.72 (d, J=10.0 Hz, 1H), 7.64-7.62 (d, J=10.0 Hz, 1H), 4.85-4.82 (m, 1H), 1.38-1.37 (m, 6H).

### Synthesis of 3-(5-(4-(3-isopropoxy-4-nitrobenzoyl)piperazin-1-yl)-1-oxoisoindolin-2-yl)piperidine-2,6-dione

Into a 25 mL round-bottom flask were added 3-(1-oxo-5-(piperazin-1-yl)isoindolin-2-yl)piperidine-2,6-dione (200.0 mg, 0.55 mmol, 1.0 eq), 3-methoxy-4-nitrobenzoic acid (123.5 mg, 0.55 mmol, 1.0 eq), HATU (312.7 mg, 0.82 mmol, 1.5 eq), DIEA (212.6 mg, 1.64 mmol, 3.0 eq) and DMF (2 mL). The reaction mixture was stirred for 2 hours at 25° C. The resulting mixture was diluted with water (10 mL) and then extracted with CH₂Cl₂ (2×10 mL). The combined organic layers were washed with brine (10 mL), dried over anhydrous Na₂SO₄. After filtration, the filtrate was concentrated under reduced pressure. The crude residue was purified by silica gel column (dichloromethane/methanol=100:1-20:1) to afford 3-(5-(4-(3-isopropoxy-4-nitrobenzoyl)piperazin-1-yl)-1-oxoisoindolin-2-yl)piperidine-2,6-dione (153 mg, 52%) as yellow solid. LC-MS: (ESI, m/z): M+1: 536.

### Synthesis of 3-(5-(4-(4-amino-3-isopropoxybenzoyl)piperazin-1-yl)-1-oxoisoindolin-2-yl)piperidine-2,6-dione

Into a 10 mL round-bottom flask were added 3-(5-(4-(3-isopropoxy-4-nitrobenzoyl)piperazin-1-yl)-1-oxoisoindolin-2-yl)piperidine-2,6-dione (153.0 mg, 0.29 mmol, 1.0 eq), Fe (79.8 mg, 1.43 mmol, 5.0 eq), NH₄Cl (152.9 mg, 2.86 mmol, 10.0 eq), water (1.5 mL) and EtOH (3.0 mL) at 25° C. The resulting mixture was stirred for 16 hours at 75° C. The reaction mixture was filtered. The filter cake was washed with EtOH (8×5 mL). The combined filtrate was collected and concentrated. The residue was washed with water (2×5 mL), and then dried under vacuum to afford 3-(5-(4-(4-amino-3-isopropoxybenzoyl)piperazin-1-yl)-1-oxoisoindolin-2-yl)piperidine-2,6-dione (80 mg, 55%) as yellow solid. LC-MS: (ESI, m/z): M+1: 506.

### Synthesis of (2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-isopropoxyphenyl)-2-neopentyl-6′-(trifluoromethyl)-1′,2′-dihydrospiro[pyrrolidine-3,3′-pyrrolo[3,2-c]pyridine]-5-carboxamide

To a solution of 3-(5-(4-(4-amino-3-isopropoxybenzoyl)piperazin-1-yl)-1-oxoisoindolin-2-yl)piperidine-2,6-dione (40.0 mg, 0.08 mmol, 1.0 eq) in MeCN (1.2 mL) were added (2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxylic acid (53.8 mg, 0.11 mmol, 1.4 eq), TCFH (44.4 mg, 0.16 mmol, 2.0 eq) and NMI (21.4 mg, 0.26 mmol, 3.3 eq). The resulting mixture was stirred for 2 hours at 25° C. The reaction was quenched with water (5 mL) and extracted with CHCl₃ (2×5 mL). The combined organic layers were washed with brine (5 mL), dried over anhydrous Na₂SO₄. After filtration, the filtrate was concentrated under reduced pressure. The residue was purified by silica gel column chromatography, eluted with dichloromethane/methanol=100:1-20:1 and then re-purified by Prep-HPLC using the following conditions: Column: XBridge C18 OBD Column, 19*150 mm, 5 μm; Mobile Phase A: Water (0.1% FA), Mobile Phase B: CH₃CN; Flow rate: 20 mL/min; Gradient: 5% B to 55% B in 9 min, 55% B; Wave Length: 254/220 nm; RT=8.6 mins. Finally, (2S,3S,4S,5R)-4-(3-chloro-2-fluorophenyl)-N-(4-(4-(2-(2,6-dioxopiperidin-3-yl)-1-oxoisoindolin-5-yl)piperazine-1-carbonyl)-2-isopropoxyphenyl)-2-neopentyl-6′-(trifluoromethyl)-1′,2′-dihydrospiro[pyrrolidine-3,3′-pyrrolo[3,2-c]pyridine]-5-carboxamide (3.19 mg, 4.1%) was obtained as a white solid. LC-MS: (ESI, m/z): M/2+1: 487. ¹HNMR (500 MHz, Methanol-d₄) δ 8.38-8.37 (d, J=5.0 Hz, 1H), 8.26 (s, 1H), 7.66-7.63 (m, 1H), 7.35 (m, 1H), 7.33 (m, 1H), 7.16 (m, 2H), 7.11 (m, 2H), 7.03 (d, J=5.0 Hz, 1H), 6.65 (s, 1H), 5.11-5.08 (m, 1H), 4.88 (m, 1H), 4.49 (m, 1H)), 4.47 (m, 1H), 4.32 (m, 1H), 3.78 (m, 3H), 3.53 (m, 2H), 3.52 (m, 2H), 2.57 (m, 1H) 2.55 (m, 4H), 2.33 (m, 2H), 1.77-1.74 (m, 2H), 1.43-1.38 (m, 4H), 1.30 (m, 4H), 0.98 (s, 9H).

### Example 92: Preparation of (2S,3S,4S,5R)-4-(3-chloro-2-fluorophenyl)-N-(4-(4-(2-(2,6-dioxopiperidin-3-yl)-1,3-dioxoisoindolin-5-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

### Synthesis of 2-(2,6-dioxopiperidin-3-yl)-5-(4-(3-methoxy-4-nitrobenzoyl)piperazin-1-yl)isoindoline-1,3-dione

Into a 25 mL round-bottom flask were added 2-(2,6-dioxopiperidin-3-yl)-5-(piperazin-1-yl)isoindoline-1,3-dione (500.0 mg, 1.3 mmol, 1.0 eq), 3-methoxy-4-nitrobenzoic acid (234.0 mg, 1.2 mmol, 0.9 eq), HATU (752.0 mg, 2.0 mmol, 1.5 eq), DIEA (511.0 mg, 4.0 mmol, 3.0 eq) and DMF (5 mL). The resulting mixture was stirred for 2 hours at 25° C. The reaction mixture was diluted with water (25 mL) and extracted with CH₂Cl₂ (2×25 mL). The combined organic layers were washed with brine (30 mL), dried over anhydrous Na₂SO₄. After filtration, the filtrate was concentrated under reduced pressure. The crude residue was purified by silica gel column (dichloromethane/methanol=100:1-30:1) to afford 2-(2,6-dioxopiperidin-3-yl)-5-(4-(3-methoxy-4-nitrobenzoyl)piperazin-1-yl)isoindoline-1,3-dione (540 mg, 78%) as yellow solid. ¹HNMR (500 MHz, DMSO-d₆) δ 11.05 (s, 1H), 8.15 (s, 1H), 7.97-7.95 (m, 2H), 7.72-7.71 (d, J=5.0 Hz, 1H), 7.42 (s, 1H), 7.36 (s, 1H), 7.27-7.25 (d, J=10.0 Hz, 1H), 7.18-7.16 (d, J=10.0 Hz, 1H), 5.09-5.06 (m, 1H), 3.96 (s, 3H), 3.80-3.79 (m, 2H), 3.62-3.61 (m, 5H), 3.53 (m, 4H), 3.15-3.12 (m, 2H), 2.89 (m, 4H), 2.73 (m, 4H), 2.61-2.55 (m, 2H).

### Synthesis of 5-(4-(4-amino-3-methoxybenzoyl)piperazin-1-yl)-2-(2,6-dioxopiperidin-3-yl)isoindoline-1,3-dione

Into a 10 mL round-bottom flask were added 2-(2,6-dioxopiperidin-3-yl)-5-(4-(3-methoxy-4-nitrobenzoyl)piperazin-1-yl)isoindoline-1,3-dione (250.0 mg, 0.5 mmol, 1.0 eq), Fe (134.0 mg, 2.4 mmol, 5.0 eq), NH₄Cl (256.0 mg, 4.8 mmol, 10.0 eq), water (2.5 mL) and EtOH (5.0 mL) at 25° C. The resulting mixture was stirred for 16 hours at 75° C. The reaction mixture was filtered. The filter cake was washed with EtOH (8×5 mL). The combined filtrate was collected and concentrated. The residue was washed with water (2×5 mL), and then dried under vacuum to afford 5-(4-(4-amino-3-methoxybenzoyl)piperazin-1-yl)-2-(2,6-dioxopiperidin-3-yl)isoindoline-1,3-dione (93 mg, 39%) as yellow solid. ¹HNMR (400 MHz, DMSO-d₆) δ 11.06 (s, 1H), 7.68-7.62 (m, 1H), 7.32-7.20 (m, 2H), 6.86-6.81 (m, 2H), 6.60-6.58 (m, 1H), 5.06-5.02 (m, 3H), 3.75-3.38 (m, 13H), 2.85-2.46 (m, 4H), 2.01-1.95 (m, 2H).

### Synthesis of (2S,3S,4S,5R)-4-(3-chloro-2-fluorophenyl)-N-(4-(4-(2-(2,6-dioxopiperidin-3-yl)-1,3-dioxoisoindolin-5-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide

To a solution of 5-(4-(4-amino-3-methoxybenzoyl)piperazin-1-yl)-2-(2,6-dioxopiperidin-3-yl)isoindoline-1,3-dione (93.0 mg, 0.2 mmol, 1.0 eq) in MeCN (4 mL) were added (2S,3S,4S,5R)-4-(3-chloro-2-fluorophenyl)-2-neopentyl-6′-(trifluoromethyl)-1′,2′-dihydrospiro[pyrrolidine-3,3′-pyrrolo[3,2-c]pyridine]-5-carboxylic acid (82.0 mg, 0.2 mmol, 1.0 eq), TCFH (106.0 mg, 0.4 mmol, 2.0 eq) and NMI (93.0 mg, 1.2 mmol, 6.0 eq). The resulting mixture was stirred for 2 hours at 25° C. The reaction was quenched with water (10 mL) and extracted with CHCl₃ (2×10 mL). The combined organic layers were washed with brine (10 mL), dried over anhydrous Na₂SO₄. After filtration, the filtrate was concentrated under reduced pressure. The residue was purified by silica gel column chromatography, eluted with dichloromethane/methanol=100:1-30:1 and then re-purified by Prep-HPLC using the following conditions: Column: XBridge C18 OBD Column, 19*150 mm, 5 μm; Mobile Phase A: Water(0.1% FA), Mobile Phase B: CH₃CN; Flow rate: 20 mL/min; Gradient: 5% B to 55% B in 9 min, 55% B; Wave Length: 254/220 nm; RT=8.5 mins. Finally, (2S,3S,4S,5R)-4-(3-chloro-2-fluorophenyl)-N-(4-(4-(2-(2,6-dioxopiperidin-3-yl)-1,3-dioxoisoindolin-5-yl)piperazine-1-carbonyl)-2-methoxyphenyl)-2-neopentyl-6′-(trifluoromethyl)-1′,2′-dihydrospiro[pyrrolidine-3,3′-pyrrolo[3,2-c]pyridine]-5-carboxamide (6.6 mg, 3.6%) was obtained as a white solid. LC-MS: (ESI, m/z): M+1: 959/960. ¹HNMR (500 MHz, Methanol-d₄) δ 8.58 (s, 1H), 8.39-8.37 (d, J=10.0 Hz, 1H), 8.25 (s, 1H), 7.71-7.69 (d, J=10.0 Hz, 1H), 7.39 (m, 2H), 7.33 (m, 2H), 7.31 (m, 1H), 7.17-7.15 (m, 2H), 7.07 (d, J=10.0 Hz, 1H)), 6.99 (s, 1H), 5.08-5.05 (m, 1H), 4.47-4.45 (m, 1H), 4.27-4.25 (m, 1H), 3.95 (s, 3H), 3.78 (m, 2H), 3.56 (m, 2H), 3.48-3.45 (m, 4H), 2.85-2.73 (m, 3H), 2.12-2.11 (m, 1H), 1.44 (m, 1H), 1.35-1.29 (m, 2H), 1.02 (s, 9H).

### Biological Example 1: In Vitro MDM2 Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53 (Lane et al). Binding of GST-MDM2 protein and p53-peptide (biotinyiated on its N-terminal) is registered by the FRET (fluorescence resonance energy transfer) between Europium (Eu)-labeled anti-GST antibody and streptavidin-conjugated Allophycocyanin (APC). Test is performed in black flat-bottom 384-well plates (Costar) in a total volume of 40 uL containing:90 nM biotinylate peptide, 160 ng/ml GST-MDM2, 20 nM streptavidin-APC (PerkinElmer Wallac), 2 nM Eu-labeled anti-GST-antibody (PerkmElmerWallac), 0.02% bovine serum albumin (BSA), 1 mM dithiothreitol (DTT) and 20 mM Tris-borate saline (TBS) buffer as follows: Add 10 ul. of GST-MDM2 (640 ng/ml working solution) in reaction buffer to each well. Add 10 uL diluted compounds (1:5 dilution in reaction buffer) to each well, mix by shaking. Add 20 uL biotinyiated p53 peptide (180 nM working solution) in reaction buffer to each well and mix on shaker. Incubate at 37° C. for 1 h. Add 2011 L streptavidin-APC and Eii-anti-GST antibody mixture (6 nM Eu-anti-GST and 60 nM streptavidin-APC working solution) in TBS buffer with 0.02% BSA, shake at room temperature for 30 minutes and read using a TRF-capable plate reader at 665 and 615 nm (Victor 5, Perk in ElmcrWallac). If not specified, the reagents were purchased from Sigma Chemical Co. Such assays, carried out with a range of doses of test compounds, allowed the determination of the MDM2 IC₅₀ of the compounds of the present invention.

### Biological Example 2: In Vitro Anti-Proliferation Assay in BCL-2-Dependent Acute Lymphoblastic Leukemia (ALL) Cell Line RS4; 11 Overexpressing Bcl-2 G101V

Cell antiproliferation was assayed by PerkinElmer ATPlite™ Luminescence Assay System. Briefly, the various test cancer cell lines were plated at a density of about 1×10⁴ cells per well in Costar 96-well plates, and were incubated with different concentrations of compounds for about 72 hours in medium supplemented with 5% FBS or 10% normal human serum(NHS). One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at ˜700 rpm. This procedure was used to lyse the cells and to stabilize the ATP. Next, 50 μL substrate solution was added to the wells and microplate was shaken for five minutes in an orbital shaker at ˜700 rpm. Finally, the luminescence was measured by a PerkinElmer TopCount® Microplate Scintillation Counter. Such assays, carried out with a range of doses of test compounds, allowed the determination of the cellular anti-antiproliferative IC₅₀ of the compounds of the present invention.

### Biological Example 3 Western Blot Assay

Western blotting is a technique that uses specific antibodies to identify proteins that have been separated based on size by gel electrophoresis. The immunoassay uses a membrane made of nitrocellulose or PVDF (polyvinylidene fluoride). The gel is placed next to the membrane and the application of an electrical current induces the proteins to migrate from the gel to the membrane. The membrane can then be further processed with antibodies specific for the target of interest and visualized using secondary antibodies and detection reagents.

In this study, RS4; 11 cells overexpressing Bcl2 G101V were plated in the 12-well plate and treated with compounds for 4 hrs at 37 C. After removal of media, cells were washed with PBS one time. The cells were then lysed directly in RIPA buffer (500ul) by pipetting and collected into Eppendorf tubes. The lysates were clarified at 13000 rpm for 15 min. The protein concentration in the supernatants was determined using the Pierce BCA protein Assay kit. The protein concentration was normalized (20ug of protein) and the samples were reduced in Laemmli's SDS-sample buffer at 70° C. in a heated block for 20 min. An equal amount of protein samples (20ul) were resolved using precast Bolt PAA gels. The protein gels were transferred onto 0.45-μm pore size nitrocellulose membranes. The membranes were blocked with non-fat dry milk (5% wt/vol) in 1×Tris-buffered saline-Tween-20 for 1 h at room temperature, and were probed with: Recombinant rabbit Anti-MDM2 antibody [EPR22256-98](ab259265) from Abcam (dilution 1:1000), Mouse monoclonal Anti-p53 antibody [DO-1]-(ab1101) from Abcam (dilution 1:2500), Recombinant rabbit monoclonal Anti-p21 antibody [EPR362](ab109520) from Abcam (dilution 1:1000), Rabbit polyclonal Anti-PUMA antibody (ab9643), Abcam (dilution 1:1000), and Mouse monoclonal Anti-GAPDH antibody [6C5](ab8245), Abcam. (1; 20,000). All antibodies were diluted in milk (5% wt/vol in TBST) and incubated overnight at 4° C. The membranes were washed three times (10 min each) in non-fat dry milk (5% wt/vol) in 1×Tris-buffered saline-Tween-20 and incubated with horse radish peroxidase (HRP)-conjugated anti-rabbit or anti-mouse secondary antibodies for 1h (1:20000) at room temperature. Following sufficient washing with TBST (twice with milk and once with only TBST), the membranes were exposed with chemiluminescent HRP substrate, and the signal was detected using FluoroChem imager (Protein Simple).

### Biological Example 4: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example 5: In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

1. A compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof: 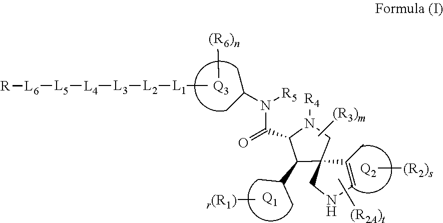
wherein
R is a small molecule (e.g., molecular weight less than about 1,500 Da, 1,200 Da, 900 Da, 500 Da or less) E3 ubiquitin ligase binding moiety that binds an E3 ubiquitin ligase;
each of Li, L₂, L₃, L₄, L₅, and L₆, independently, is absent, a bond, (CRₐR_{b})ₚ, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{g}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, bivalent alkyl, bivalent alkenyl, bivalent alkynyl, bivalent cycloalkyl, bivalent cycloalkenyl, bivalent heterocycloalkyl, bivalent spirocycloalkyl, bivalent fused-carbocyclic, bivalent bridged-carbocyclic, bivalent heterocycloalkenyl, bivalent spiro-heterocyclic, bivalent fused-heterocyclic, bivalent bridged-heterocyclic, bivalent aryl, or bivalent heteroaryl, each of which is independently optionally substituted with one or more Rd;
each of Q₁, Q₂, and Q₃, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more Rd;
each of RI, R₂, R_{2A}, R₃, R₄, R₅, and R₆, independently, is H, D, alkyl, spiroalkyl, alkenyl, alkynyl, halo, nitro, oxo, cyano, —ORₐ, —SRₐ, -alkyl-Rₐ, -alkyl-O—Rₐ, —P(O)(Rₐ)(R_{b}), —P(O)(ORₐ)(OR_{b}), -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
each Rₐ, R_{b}, R_{c} and R_{d}, independently, is H, D, alkyl, spiroalkyl, alkenyl, alkynyl, halo, cyano, amine, nitro, hydroxy, ═O, -alkyl-O—Rₑ, -alkyl-O—P(O)(OH)(OH), C(O)NHOH, C(O)OH, —C(O)O—Rₑ, —C(O)—Rₑ, C(O)NH₂, alkoxy, alkoxyalkyl, —O—Rₑ, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, each of which is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), —C(O)NHOH, —C(O)O—R_{f}, —OC(O)—R_{f}, —C(O)—R_{f}, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, each of which is independently optionally substituted with one or more R_{f}; and
R_{f} is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl, heterocycloalkyl, heterocycloalkenyl, aryl, or heteroaryl, each optionally substituted with one or more R_{d};
two of R₂ groups, taken together with the atom to which they are attached, may optionally form a cycloalkyl, heterocycloalkyl, heterocycloalkenyl, aryl, or heteroaryl, each optionally substituted with one or more R_{d};
two of R₃ groups, taken together with the atom to which they are attached, may optionally form a cycloalkyl, heterocycloalkyl, heterocycloalkenyl, aryl, or heteroaryl, each optionally substituted with one or more R_{d};
two of R₆ groups, taken together with the atom to which they are attached, may optionally form a cycloalkyl, heterocycloalkyl, heterocycloalkenyl, aryl, or heteroaryl, each optionally substituted with one or more R_{d};
R₃ and R₄ groups, taken together with the atom to which they are attached, may optionally form a cycloalkyl, heterocycloalkyl, heterocycloalkenyl, aryl, or heteroaryl, each optionally substituted with one or more R_{d};
R₄ and R₅ groups, taken together with the atom to which they are attached, may optionally form a cycloalkyl, heterocycloalkyl, heterocycloalkenyl, aryl, or heteroaryl, each optionally substituted with one or more R_{d}; and
R₅ and R₆ groups, taken together with the atom to which they are attached, may optionally form a cycloalkyl, heterocycloalkyl, heterocycloalkenyl, aryl, or heteroaryl, each optionally substituted with one or more R_{d};
two of R_{d} groups, taken together with the atom to which they are attached, may optionally form a cycloalkyl, heterocycloalkyl, heterocycloalkenyl, aryl, or heteroaryl, each optionally substituted with one or more Rₑ;
two of Rₑ groups, taken together with the atom to which they are attached, may optionally form a cycloalkyl, heterocycloalkyl, heterocycloalkenyl, aryl, or heteroaryl, each optionally substituted with one or more R_{f}; and
each of m, n, r, s, and t, independently, is 0, 1, 2, or 3;
each of p and q, independently, is 0, 1, 2, 3, or 4.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (2):

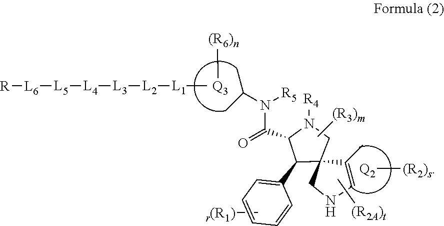

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (3): 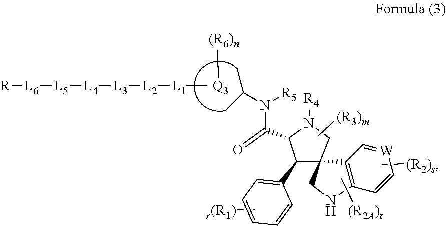
wherein
W is CH or N.

4. The compound according to claim 1 or an N-oxide thereof, or a pharmaceutically acceptable salt, solvate, polymorph, tautomer, stereoisomer, an isotopic form, or a prodrug thereof, wherein said E3 ubiquitin ligase is Cereblon, Von Hippel-Lindau, mouse double-minute homolog2, or IAP.

5. The compound according to claim 4 or an N-oxide thereof, or a pharmaceutically acceptable salt, solvate, polymorph, tautomer, stereoisomer, an isotopic form, or a prodrug thereof, wherein said E3 ubiquitin ligase is Cereblon.

6. The compound according to claim 5 or an N-oxide thereof, or a pharmaceutically acceptable salt, solvate, polymorph, tautomer, stereoisomer, an isotopic form, or a prodrug thereof, wherein the compound is represented by Formula (4): 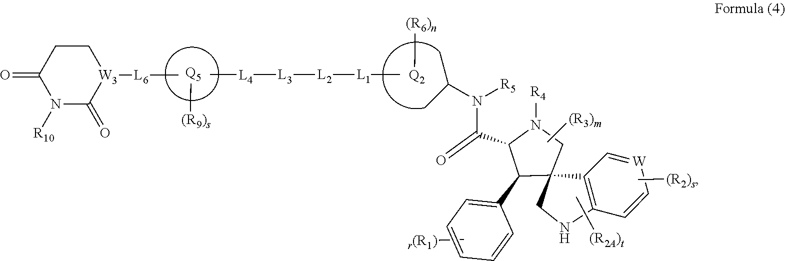
wherein
R₁₀ is H, D, -alkyl-O—P(O)(Rₐ)(R_{b}), or -alkyl-OC(O)—Rₐ;
W₃ is N or CH;
L₆ is absent, NH, CONH, or O;
Q₅ is absent, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
R₉ is absent, H, D, alkyl, spiroalkyl, alkenyl, alkynyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, each of which is independently optionally substituted with one or more R_{d};
R₉ and L₄ groups, taken together with the atom to which they are attached, may optionally form a cycloalkyl, heterocycloalkyl, heterocycloalkenyl, aryl, or heteroaryl, each optionally substituted with one or more R_{d}; and
s is 0, 1, 2, 3, or 4.

7. The compound according to claim 6 or an N-oxide thereof, or a pharmaceutically acceptable salt, solvate, polymorph, tautomer, stereoisomer, an isotopic form, or a prodrug thereof, wherein the compound is represented by Formula (5): 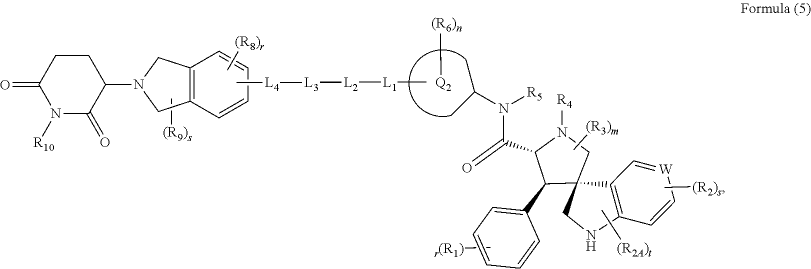
wherein
R₈ is absent, H, D, alkyl, alkenyl, alkynyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, each of which is independently optionally substituted with one or more R_{d};
R₈ and L₄ groups, taken together with the atom to which they are attached, may optionally form a cycloalkyl, heterocycloalkyl, heterocycloalkenyl, aryl, or heteroaryl, each optionally substituted with one or more R_{d}; and
r is 0, 1, 2, 3, or 4.

8. A pharmaceutical composition comprising a compound of claim 1, or an N-oxide thereof, or a pharmaceutically acceptable salt, solvate, polymorph, tautomer, stereoisomer, an isotopic form, or a prodrug of said compound or N-oxide thereof, and a pharmaceutically acceptable diluent or carrier.

9. A method of treating a neoplastic disease, autoimmune disease, and inflammatory disorder, comprising administering to a subject in need thereof an effective amount of a compound of claim 1, or an N-oxide thereof, or a pharmaceutically acceptable salt, solvate, polymorph, tautomer, stereoisomer, an isotopic form, or a prodrug of said compound, or N-oxide thereof.
